(19) United States
(12) Patent Application Publication
(10) Pub. No.: US 2025/0188471 A1
(43) Pub. Date: Jun. 12, 2025

(54) NONCOVALENTLY BRANCHED OLIGONUCLEOTIDE COMPOSITIONS

(71) Applicant: Atalanta Therapeutics, Inc., Boston, MA (US)

(72) Inventors: Matthew HASSLER, Boston, MA (US); Garth A. KINBERGER, Hyde Park, MA (US); Alexander L. PRINZEN, Boston, MA (US)

(21) Appl. No.: 18/845,980

(22) PCT Filed: Mar. 10, 2023
(86) PCT No.: PCT/US2023/064127

§ 371 Date: Sep. 11, 2024

Related U.S. Application Data
(60) Provisional application No. 63/318,943, filed on Mar. 11, 2022.

(51) Int. Cl. C12N 15/113; A61K 31/713

(52) U.S. Cl. CPC C12N 15/1137; A61K 31/713; C12N 2310/14; C12N 2310/321; C12N 2310/322; C12N 2310/3231; C12N 2310/34; C12N 2310/3513; C12N 2310/3515; C12N 2310/3519; C12N 2310/51; C12N 2310/52

(57) ABSTRACT

The present disclosure provides noncovalently-linked multimeric oligonucleotides that may exhibit efficient and specific tissue distribution, cellular uptake, minimum immune response and off-target effects. Additionally, the disclosure provides methods for delivering the siRNA molecule of the disclosure to the central nervous system of a subject, such as a subject identified as having a need for gene silencing.

### TECHNICAL FIELD

The present disclosure relates to noncovalently branched, multimeric oligonucleotides, as well as methods of using the same to modulate gene expression.

### BACKGROUND

Therapeutic oligonucleotides are effective tools for a variety of applications, including the inhibition of gene function. An example of such inhibition is RNA interference (RNAi). The promise of RNAi as a general therapeutic strategy, however, depends on the ability to deliver small RNAs to a wide range of tissues. There remains a need for additional siRNA, and therapeutic oligonucleotides in general, that exhibit minimal immune response and off-target effects, efficient cellular uptake without formulation, prolonged half-life, and efficient and specific tissue distribution.

### SUMMARY OF THE INVENTION

The present disclosure provides noncovalently branched, multimeric oligonucleotides that may exhibit improvement in distribution, in vivo efficacy, and safety.

In a first aspect, the disclosure provides a multimeric oligonucleotide having from 2 to 8 siRNA molecules (e.g., 2, 3, 4, 5, 6, 7, or 8 siRNA molecules) that are joined by way of a noncovalent interaction, wherein each siRNA molecule, independently, contains a noncovalent binding moiety allowing the siRNA molecule to noncovalently bind to at least one other siRNA molecule in the multimeric oligonucleotide. In some embodiments, each siRNA molecule is, independently, attached to the noncovalent binding moiety by way of a linker.

In some embodiments, at least one noncovalent binding moiety is a nucleic acid or a nucleic acid analog. In some embodiments, the noncovalent interaction is nucleic acid hybridization.

In some embodiments, the multimeric oligonucleotide has the following structure:

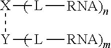

wherein X and Y are each, independently, a nucleic acid or a nucleic acid analog;
each of m and n is, independently, 1, 2, 3, or 4;
each L is, independently, absent or a linker;
each RNA is, independently, an siRNA molecule;
wherein X and Y have complementarity sufficient to hybridize to one another; and
--- represents a hybridization interaction between X and Y.

In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, the sum of m and n is 7. In some embodiments, the sum of m and n is 8.

In some embodiments, the nucleic acid contains one or more DNA nucleosides. In some embodiments, the nucleic acid contains one more RNA nucleosides. In some embodiments, the nucleic acid contains one more LNA nucleosides. In some embodiments, the nucleic acid contains only LNA nucleosides. In some embodiments, the nucleic acid analog contains a PNA (peptide nucleic acid), an LNA (locked nucleic acid), a GNA (glycol nucleic acid), a TNA (threose nucleic acid), an HNA (hexitol nucleic acid), a morpholino oligomer, or a combination thereof.

In some embodiments, each nucleic acid has from 10 to 100 nucleosides. In some embodiments, each nucleic acid has from 10 to 90 nucleosides. In some embodiments, each nucleic acid has from 10 to 80 nucleosides. In some embodiments, each nucleic acid has from 10 to 70 nucleosides. In some embodiments, each nucleic acid has from 10 to 60 nucleosides. In some embodiments, each nucleic acid has from 10 to 50 nucleosides. In some embodiments, each nucleic acid has from 10 to 40 nucleosides. In some embodiments, each nucleic acid has from 10 to 30 nucleosides. In some embodiments, each nucleic acid has from 15 to 30 nucleosides.

In some embodiments, the duplex formed between X and Y has a length of 1 to 20 contiguous nucleotides. In some embodiments, the duplex formed between X and Y has a length of 1 to 10 contiguous nucleotides. In some embodiments, the duplex formed between X and Y has a length of 1 to 5 contiguous nucleotides. In some embodiments, the duplex formed between X and Y has a length of 5 to 10 contiguous nucleotides.

In some embodiments, the duplex formed between X and Y has a length of 2 contiguous nucleotides. In some embodiments, the duplex formed between X and Y has a length of 2 contiguous nucleotides. In some embodiments, the duplex formed between X and Y has a length of 3 contiguous nucleotides. In some embodiments, the duplex formed between X and Y has a length of 4 contiguous nucleotides. In some embodiments, the duplex formed between X and Y has a length of 5 contiguous nucleotides. In some embodiments, the duplex formed between X and Y has a length of 6 contiguous nucleotides. In some embodiments, the duplex formed between X and Y has a length of 7 contiguous nucleotides. In some embodiments, the duplex formed between X and Y has a length of 8 contiguous nucleotides. In some embodiments, the duplex formed between X and Y has a length of 9 contiguous nucleotides. In some embodiments, the duplex formed between X and Y has a length of 10 contiguous nucleotides.

In some embodiments, the multimeric oligonucleotide contains from 2 to 7 siRNA molecules. In some embodiments, the multimeric oligonucleotide contains from 2 to 6 siRNA molecules. In some embodiments, the multimeric oligonucleotide contains from 2 to 5 siRNA molecules. In some embodiments, the multimeric oligonucleotide contains from 2 to 4 siRNA molecules. In some embodiments, the multimeric oligonucleotide contains 2 or 3 siRNA molecules. In some embodiments, the multimeric oligonucleotide contains 2 siRNA molecules.

In some embodiments, each siRNA molecule is, independently, attached to the noncovalent binding moiety by way of a linker.

In some embodiments, the multimeric oligonucleotide has the following structure:

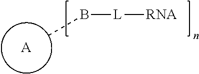

wherein A is a host molecule;
each B is, independently, a guest moiety that noncovalently binds to the host molecule;
each --- is a noncovalent interaction between the host molecule and the guest moiety;
each L is, independently, absent or a linker;
each RNA is, independently, an siRNA molecule; and
n is an integer from 2-8.

In some embodiments, the multimeric oligonucleotide is of the following structure:

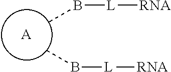

In some embodiments, the multimeric oligonucleotide is of the following structure:

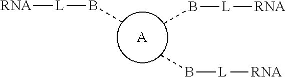

In some embodiments, the multimeric oligonucleotide is of the following structure:

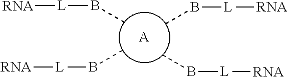

In some embodiments, the noncovalent interaction is an electrostatic interaction, a Lewis acid-base interaction, a hydrogen-bonding interaction, a hydrophobic interaction, a Van der Waals interaction, a π-effect, or a protein ligand interaction, or a combination thereof.

In some embodiments, A is a macrocyclic ring. In some embodiments, A is a macrocyclic oligosaccharide. In some embodiments, A is a cyclodextrin. In a particular embodiment, A is β-cyclodextrin.

In some embodiments, each B is, independently:

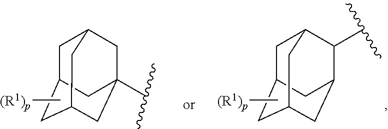

wherein each R¹ is, independently, hydroxyl, optionally substituted amino, optionally substituted alkoxy, optionally substituted C₁₋₆ alkyl, or —CO₂H; and
p is 0, 1, 2, 3, 4, or 5.

In some embodiments, each B is, independently:

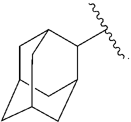 [SMILES: *C1C2CC3CC(C2)CC1C3]

In some embodiments, each B is, independently:

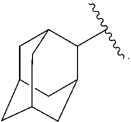 [SMILES: *C1C2CC3CC(C2)CC1C3]

In some embodiments, p is 0. In some embodiments, p is 1. In some embodiments, p is 2.

In some embodiments, R¹ is hydroxyl. In some embodiments, each B is, independently:

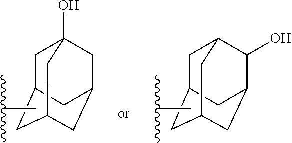

wherein

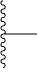

represents a point of attachment to L and is substituted at any hydrogen atom in B.

In some embodiments, R¹ is optionally substituted amino. In some embodiments, R¹ is —NH₂ In some embodiments, each B is:

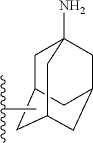

wherein

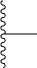

represents a point of attachment to L and is substituted at any hydrogen atom in B.

In some embodiments, R¹ is —CO₂H. In some embodiments, each B is:

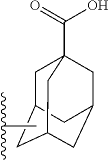

wherein

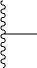

represents a point of attachment to L and is substituted at any hydrogen atom in B.

In some embodiments, each B is:

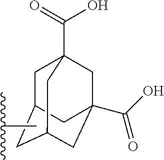

wherein

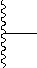

represents a point of attachment to L and is substituted at any hydrogen atom in B.

In some embodiments, R¹ is optionally substituted C₁₋₆ alkyl. In some embodiments, each B is:

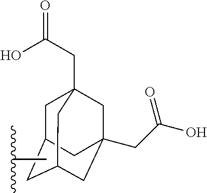

wherein

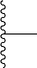

represents a point of attachment to L and is substituted at any hydrogen atom in B.

In some embodiments, A is a protein. In some embodiments, A is a biotin-binding protein.

In some embodiments, A is avidin, streptavidin, NeutrAvidin, Bradavidin II, hoefavidin, rhizavidin, Tamavidin 2, Shwanavidin, Switchavidin, Zebavidin, or Strep-Tactin®.

In some embodiments, each B is, independently, a ligand that binds to a protein.

In some embodiments, each B is, independently, a peptide. In some embodiments, each B has the peptide sequence WSHPQFEK (SEQ ID NO: 1).

In some embodiments, each B is, independently:

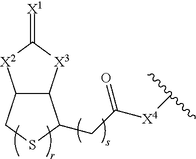

wherein
X¹, X², X³ and X⁴ are each, independently, O or NR²;
each R₂ is, independently, H or optionally substituted C₁₋₆ alkyl;
r is 0 or 1; and
s is 1, 2, 3, 4, 5, or 6.

In some embodiments, X¹ is O. In some embodiments, X¹ is NR². In some embodiments, X² is NR². In some embodiments, X³ is NR². In some embodiments, R² is H. In some embodiments, R² is optionally substituted C₁₋₆ alkyl. In some embodiments, R² is ethyl. In some embodiments, X² is O. In some embodiments, X³ is O. In some embodiments, r is 0. In some embodiments, r is 1. In some embodiments, s is 1. In some embodiments, s is 2. In some embodiments, s is 3. In some embodiments, s is 4. In some embodiments, s is 5. In some embodiments, s is 6.

In some embodiments, A is a transferrin binding protein. In some embodiments, the transferrin binding protein is TbpA or TbpB. In some embodiments, B is transferrin, or an analog thereof, or an isoform thereof.

In some embodiments, the protein is an antibody. In some embodiments, the antibody is capable of binding to two or more antigens. In some embodiments, B is an antigen.

In some embodiments, A is a metal ion. In some embodiments, A is an iron ion, a copper ion, a magnesium ion, a manganese ion, a gadolinium ion, or a zinc ion. In some embodiments, A is an iron ion. In some embodiments, A is copper (I), copper (II), magnesium (II), manganese (II), manganese (III), manganese (IV), manganese (V), manganese (VI), gadolinium (III), or zinc (II). In particular embodiments, A is iron (III).

In some embodiments, each B is, independently:

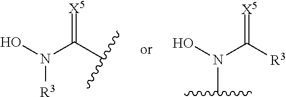

wherein
R³ is hydrogen, optionally substituted C₁-C₆ alkyl, optionally substituted C₁-C₆ alkenyl, optionally substituted C₁-C₆ alkynyl;
X⁵ is O, S, or NR⁴; and
R⁴ is H or optionally substituted C₁₋₆ alkyl.

In some embodiments, each B is, independently:

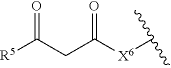

wherein
X⁶ is absent, O, or NR⁶;
R⁵ is optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, optionally substituted C₁₋₈ heteroaryl, —N(R^{5A}) 2, or —OR^{5B};
each R^{5A} is independently hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl;
R^{5B} is hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl; and
R⁶, if present, is hydrogen or optionally substituted C₁₋₆ alkyl.

In some embodiments, each B is, independently, a di-, tri-, tetra-, or poly-carboxylic acid. In some embodiments, B is EDTA.

In some embodiments, the linker is one or more contiguous subunits of an ethylene glycol, alkyl, carbohydrate, block copolymer, peptide, RNA, and DNA.

In some embodiments, the one or more contiguous subunits is 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, each siRNA molecule independently contains an antisense strand and a sense strand having complementarity to the antisense strand, wherein the antisense strand is from 10 to 30 nucleotides in length and has complementarity sufficient to hybridize to a region within a target mRNA. In some embodiments of any one of the foregoing aspects, the length of the antisense strand is 10 to 30 (e.g., 12 to 28, 14 to 26, 16 to 24, or 18 to 22) nucleotides. In some embodiments, the length of the antisense strand is 15 to 25 (e.g., 16 to 24, 17 to 23, 18 to 22, or 19 to 21) nucleotides. In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides. In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments of any one of the foregoing aspects, the length of the sense strand is between 12 and 20 (e.g., between 13 and 19, between 14 and 18, or between 15 and 17) nucleotides. In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, the antisense strand is 18 nucleotides in length and the sense strand is 14 nucleotides in length.

In some embodiments, the antisense strand is 18 nucleotides in length and the sense strand is 15 nucleotides in length.

In some embodiments, the antisense strand is 18 nucleotides in length and the sense strand is 16 nucleotides in length.

In some embodiments, the antisense strand is 18 nucleotides in length and the sense strand is 17 nucleotides in length.

In some embodiments, the antisense strand is 18 nucleotides in length and the sense strand is 18 nucleotides in length.

In some embodiments, the antisense strand is 19 nucleotides in length and the sense strand is 14 nucleotides in length.

In some embodiments, the antisense strand is 19 nucleotides in length and the sense strand is 15 nucleotides in length.

In some embodiments, the antisense strand is 19 nucleotides in length and the sense strand is 16 nucleotides in length.

In some embodiments, the antisense strand is 19 nucleotides in length and the sense strand is 17 nucleotides in length.

In some embodiments, the antisense strand is 19 nucleotides in length and the sense strand is 18 nucleotides in length.

In some embodiments, the antisense strand is 19 nucleotides in length and the sense strand is 19 nucleotides in length.

In some embodiments, the antisense strand is 20 nucleotides in length and the sense strand is 14 nucleotides in length.

In some embodiments, the antisense strand is 20 nucleotides in length and the sense strand is 15 nucleotides in length.

In some embodiments, the antisense strand is 20 nucleotides in length and the sense strand is 16 nucleotides in length.

In some embodiments, the antisense strand is 20 nucleotides in length and the sense strand is 17 nucleotides in length.

In some embodiments, the antisense strand is 20 nucleotides in length and the sense strand is 18 nucleotides in length.

In some embodiments, the antisense strand is 20 nucleotides in length and the sense strand is 19 nucleotides in length.

In some embodiments, the antisense strand is 20 nucleotides in length and the sense strand is 20 nucleotides in length.

In some embodiments, the antisense strand is 21 nucleotides in length and the sense strand is 14 nucleotides in length.

In some embodiments, the antisense strand is 21 nucleotides in length and the sense strand is 15 nucleotides in length.

In some embodiments, the antisense strand is 21 nucleotides in length and the sense strand is 16 nucleotides in length.

In some embodiments, the antisense strand is 21 nucleotides in length and the sense strand is 17 nucleotides in length.

In some embodiments, the antisense strand is 21 nucleotides in length and the sense strand is 18 nucleotides in length.

In some embodiments, the antisense strand is 21 nucleotides in length and the sense strand is 19 nucleotides in length.

In some embodiments, the antisense strand is 21 nucleotides in length and the sense strand is 20 nucleotides in length.

In some embodiments, the antisense strand is 21 nucleotides in length and the sense strand is 21 nucleotides in length.

In some embodiments, the antisense strand is 22 nucleotides in length and the sense strand is 14 nucleotides in length.

In some embodiments, the antisense strand is 22 nucleotides in length and the sense strand is 15 nucleotides in length.

In some embodiments, the antisense strand is 22 nucleotides in length and the sense strand is 16 nucleotides in length.

In some embodiments, the antisense strand is 22 nucleotides in length and the sense strand is 17 nucleotides in length.

In some embodiments, the antisense strand is 22 nucleotides in length and the sense strand is 18 nucleotides in length.

In some embodiments, the antisense strand is 22 nucleotides in length and the sense strand is 19 nucleotides in length.

In some embodiments, the antisense strand is 22 nucleotides in length and the sense strand is 20 nucleotides in length.

In some embodiments, the antisense strand is 22 nucleotides in length and the sense strand is 21 nucleotides in length.

In some embodiments, the antisense strand is 22 nucleotides in length and the sense strand is 22 nucleotides in length.

In some embodiments, the antisense strand is 23 nucleotides in length and the sense strand is 14 nucleotides in length.

In some embodiments, the antisense strand is 23 nucleotides in length and the sense strand is 15 nucleotides in length.

In some embodiments, the antisense strand is 23 nucleotides in length and the sense strand is 16 nucleotides in length.

In some embodiments, the antisense strand is 23 nucleotides in length and the sense strand is 17 nucleotides in length.

In some embodiments, the antisense strand is 23 nucleotides in length and the sense strand is 18 nucleotides in length.

In some embodiments, the antisense strand is 23 nucleotides in length and the sense strand is 19 nucleotides in length.

In some embodiments, the antisense strand is 23 nucleotides in length and the sense strand is 20 nucleotides in length.

In some embodiments, the antisense strand is 23 nucleotides in length and the sense strand is 21 nucleotides in length.

In some embodiments, the antisense strand is 23 nucleotides in length and the sense strand is 22 nucleotides in length.

In some embodiments, the antisense strand is 23 nucleotides in length and the sense strand is 23 nucleotides in length.

In some embodiments, the antisense strand is 24 nucleotides in length and the sense strand is 14 nucleotides in length.

In some embodiments, the antisense strand is 24 nucleotides in length and the sense strand is 15 nucleotides in length.

In some embodiments, the antisense strand is 24 nucleotides in length and the sense strand is 16 nucleotides in length.

In some embodiments, the antisense strand is 24 nucleotides in length and the sense strand is 17 nucleotides in length.

In some embodiments, the antisense strand is 24 nucleotides in length and the sense strand is 18 nucleotides in length.

In some embodiments, the antisense strand is 24 nucleotides in length and the sense strand is 19 nucleotides in length.

In some embodiments, the antisense strand is 24 nucleotides in length and the sense strand is 20 nucleotides in length.

In some embodiments, the antisense strand is 24 nucleotides in length and the sense strand is 21 nucleotides in length.

In some embodiments, the antisense strand is 24 nucleotides in length and the sense strand is 22 nucleotides in length.

In some embodiments, the antisense strand is 24 nucleotides in length and the sense strand is 23 nucleotides in length.

In some embodiments, the antisense strand is 24 nucleotides in length and the sense strand is 24 nucleotides in length.

In some embodiments, the antisense strand is 25 nucleotides in length and the sense strand is 14 nucleotides in length.

In some embodiments, the antisense strand is 25 nucleotides in length and the sense strand is 15 nucleotides in length.

In some embodiments, the antisense strand is 25 nucleotides in length and the sense strand is 16 nucleotides in length.

In some embodiments, the antisense strand is 25 nucleotides in length and the sense strand is 17 nucleotides in length.

In some embodiments, the antisense strand is 25 nucleotides in length and the sense strand is 18 nucleotides in length.

In some embodiments, the antisense strand is 25 nucleotides in length and the sense strand is 19 nucleotides in length.

In some embodiments, the antisense strand is 25 nucleotides in length and the sense strand is 20 nucleotides in length.

In some embodiments, the antisense strand is 25 nucleotides in length and the sense strand is 21 nucleotides in length.

In some embodiments, the antisense strand is 25 nucleotides in length and the sense strand is 22 nucleotides in length.

In some embodiments, the antisense strand is 25 nucleotides in length and the sense strand is 23 nucleotides in length.

In some embodiments, the antisense strand is 25 nucleotides in length and the sense strand is 24 nucleotides in length.

In some embodiments, the antisense strand is 25 nucleotides in length and the sense strand is 25 nucleotides in length.

In some embodiments, the antisense strand is 26 nucleotides in length and the sense strand is 14 nucleotides in length.

In some embodiments, the antisense strand is 26 nucleotides in length and the sense strand is 15 nucleotides in length.

In some embodiments, the antisense strand is 26 nucleotides in length and the sense strand is 16 nucleotides in length.

In some embodiments, the antisense strand is 26 nucleotides in length and the sense strand is 17 nucleotides in length.

In some embodiments, the antisense strand is 26 nucleotides in length and the sense strand is 18 nucleotides in length.

In some embodiments, the antisense strand is 26 nucleotides in length and the sense strand is 19 nucleotides in length.

In some embodiments, the antisense strand is 26 nucleotides in length and the sense strand is 20 nucleotides in length.

In some embodiments, the antisense strand is 26 nucleotides in length and the sense strand is 21 nucleotides in length.

In some embodiments, the antisense strand is 26 nucleotides in length and the sense strand is 22 nucleotides in length.

In some embodiments, the antisense strand is 26 nucleotides in length and the sense strand is 23 nucleotides in length.

In some embodiments, the antisense strand is 26 nucleotides in length and the sense strand is 24 nucleotides in length.

In some embodiments, the antisense strand is 26 nucleotides in length and the sense strand is 25 nucleotides in length.

In some embodiments, the antisense strand is 26 nucleotides in length and the sense strand is 26 nucleotides in length.

In some embodiments, the antisense strand is 27 nucleotides in length and the sense strand is 14 nucleotides in length.

In some embodiments, the antisense strand is 27 nucleotides in length and the sense strand is 15 nucleotides in length.

In some embodiments, the antisense strand is 27 nucleotides in length and the sense strand is 16 nucleotides in length.

In some embodiments, the antisense strand is 27 nucleotides in length and the sense strand is 17 nucleotides in length.

In some embodiments, the antisense strand is 27 nucleotides in length and the sense strand is 18 nucleotides in length.

In some embodiments, the antisense strand is 27 nucleotides in length and the sense strand is 19 nucleotides in length.

In some embodiments, the antisense strand is 27 nucleotides in length and the sense strand is 20 nucleotides in length.

In some embodiments, the antisense strand is 27 nucleotides in length and the sense strand is 21 nucleotides in length.

In some embodiments, the antisense strand is 27 nucleotides in length and the sense strand is 22 nucleotides in length.

In some embodiments, the antisense strand is 27 nucleotides in length and the sense strand is 23 nucleotides in length.

In some embodiments, the antisense strand is 27 nucleotides in length and the sense strand is 24 nucleotides in length.

In some embodiments, the antisense strand is 27 nucleotides in length and the sense strand is 25 nucleotides in length.

In some embodiments, the antisense strand is 27 nucleotides in length and the sense strand is 26 nucleotides in length.

In some embodiments, the antisense strand is 27 nucleotides in length and the sense strand is 27 nucleotides in length.

In some embodiments, the antisense strand is 28 nucleotides in length and the sense strand is 14 nucleotides in length.

In some embodiments, the antisense strand is 28 nucleotides in length and the sense strand is 15 nucleotides in length.

In some embodiments, the antisense strand is 28 nucleotides in length and the sense strand is 16 nucleotides in length.

In some embodiments, the antisense strand is 28 nucleotides in length and the sense strand is 17 nucleotides in length.

In some embodiments, the antisense strand is 28 nucleotides in length and the sense strand is 18 nucleotides in length.

In some embodiments, the antisense strand is 28 nucleotides in length and the sense strand is 19 nucleotides in length.

In some embodiments, the antisense strand is 28 nucleotides in length and the sense strand is 20 nucleotides in length.

In some embodiments, the antisense strand is 28 nucleotides in length and the sense strand is 21 nucleotides in length.

In some embodiments, the antisense strand is 28 nucleotides in length and the sense strand is 22 nucleotides in length.

In some embodiments, the antisense strand is 28 nucleotides in length and the sense strand is 23 nucleotides in length.

In some embodiments, the antisense strand is 28 nucleotides in length and the sense strand is 24 nucleotides in length.

In some embodiments, the antisense strand is 28 nucleotides in length and the sense strand is 25 nucleotides in length.

In some embodiments, the antisense strand is 28 nucleotides in length and the sense strand is 26 nucleotides in length.

In some embodiments, the antisense strand is 28 nucleotides in length and the sense strand is 27 nucleotides in length.

In some embodiments, the antisense strand is 28 nucleotides in length and the sense strand is 28 nucleotides in length.

In some embodiments, the antisense strand is 29 nucleotides in length and the sense strand is 14 nucleotides in length.

In some embodiments, the antisense strand is 29 nucleotides in length and the sense strand is 15 nucleotides in length.

In some embodiments, the antisense strand is 29 nucleotides in length and the sense strand is 16 nucleotides in length.

In some embodiments, the antisense strand is 29 nucleotides in length and the sense strand is 17 nucleotides in length.

In some embodiments, the antisense strand is 29 nucleotides in length and the sense strand is 18 nucleotides in length.

In some embodiments, the antisense strand is 29 nucleotides in length and the sense strand is 19 nucleotides in length.

In some embodiments, the antisense strand is 29 nucleotides in length and the sense strand is 20 nucleotides in length.

In some embodiments, the antisense strand is 29 nucleotides in length and the sense strand is 21 nucleotides in length.

In some embodiments, the antisense strand is 29 nucleotides in length and the sense strand is 22 nucleotides in length.

In some embodiments, the antisense strand is 29 nucleotides in length and the sense strand is 23 nucleotides in length.

In some embodiments, the antisense strand is 29 nucleotides in length and the sense strand is 24 nucleotides in length.

In some embodiments, the antisense strand is 29 nucleotides in length and the sense strand is 25 nucleotides in length.

In some embodiments, the antisense strand is 29 nucleotides in length and the sense strand is 26 nucleotides in length.

In some embodiments, the antisense strand is 29 nucleotides in length and the sense strand is 27 nucleotides in length.

In some embodiments, the antisense strand is 29 nucleotides in length and the sense strand is 28 nucleotides in length.

In some embodiments, the antisense strand is 29 nucleotides in length and the sense strand is 29 nucleotides in length.

In some embodiments, the antisense strand is 30 nucleotides in length and the sense strand is 14 nucleotides in length.

In some embodiments, the antisense strand is 30 nucleotides in length and the sense strand is 15 nucleotides in length.

In some embodiments, the antisense strand is 30 nucleotides in length and the sense strand is 16 nucleotides in length.

In some embodiments, the antisense strand is 30 nucleotides in length and the sense strand is 17 nucleotides in length.

In some embodiments, the antisense strand is 30 nucleotides in length and the sense strand is 18 nucleotides in length.

In some embodiments, the antisense strand is 30 nucleotides in length and the sense strand is 19 nucleotides in length.

In some embodiments, the antisense strand is 30 nucleotides in length and the sense strand is 20 nucleotides in length.

In some embodiments, the antisense strand is 30 nucleotides in length and the sense strand is 21 nucleotides in length.

In some embodiments, the antisense strand is 30 nucleotides in length and the sense strand is 22 nucleotides in length.

In some embodiments, the antisense strand is 30 nucleotides in length and the sense strand is 23 nucleotides in length.

In some embodiments, the antisense strand is 30 nucleotides in length and the sense strand is 24 nucleotides in length.

In some embodiments, the antisense strand is 30 nucleotides in length and the sense strand is 25 nucleotides in length.

In some embodiments, the antisense strand is 30 nucleotides in length and the sense strand is 26 nucleotides in length.

In some embodiments, the antisense strand is 30 nucleotides in length and the sense strand is 27 nucleotides in length.

In some embodiments, the antisense strand is 30 nucleotides in length and the sense strand is 28 nucleotides in length.

In some embodiments, the antisense strand is 30 nucleotides in length and the sense strand is 29 nucleotides in length.

In some embodiments, the antisense strand is 30 nucleotides in length and the sense strand is 30 nucleotides in length.

In some embodiments, the antisense strand has a structure represented by Formula I, wherein Formula I is, in the 5′-to-3′ direction:

A-B-(A′)ⱼ-C-P²-D-P¹-(C′-P¹)ₖ-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

A-B-(A′)ⱼ-C-P²-D-P¹-(C-P¹)ₖ-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

A-(A′)ⱼ-C-P²-B-(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

A-Bⱼ-E-Bₖ-E-F-Gₗ-D-P¹-C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

H-Bₘ—Iₙ-A′-Bₒ—H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
o is an integer from 1 to 7 (e.g., 1, 2, 3, 4, 5, 6, or 7).

In some embodiments, the sense strand has a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, X, XI, XII, XIII, XIV, XV, or XVI:

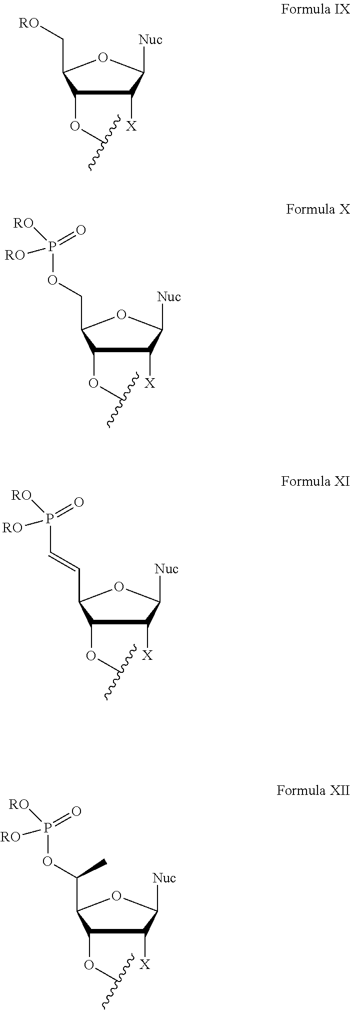

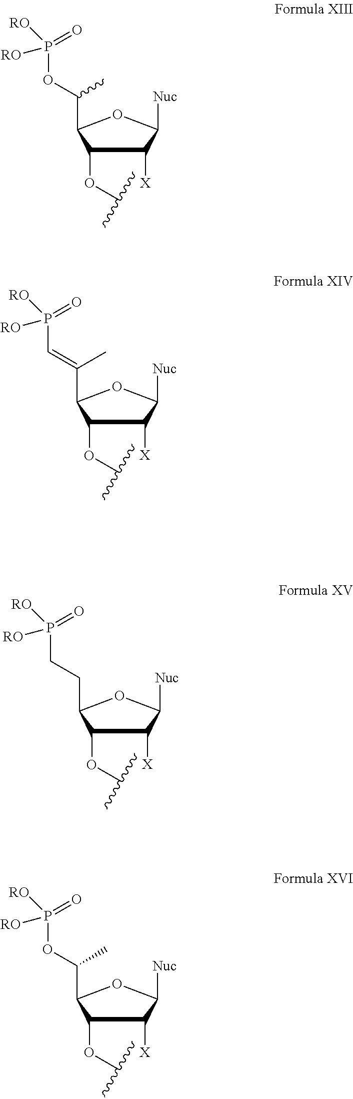

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydrogen, or a cation (e.g., a monovalent cation).

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also contains a hydrophobic moiety at the 5′ or the 3′ end of the siRNA molecule.

In some embodiments, the hydrophobic moiety is cholesterol, vitamin D, or tocopherol.

In another aspect, the disclosure provides a composition of siRNA molecules containing the multimeric oligonucleotide of any of the preceding aspects or embodiments of the disclosure, wherein the multimeric oligonucleotide is present in the composition with a purity of 50% or more. In some embodiments, the multimeric oligonucleotide is present in the composition with a purity of 75% or more. In some embodiments, the multimeric oligonucleotide is present in the composition with a purity of 80% or more. In some embodiments, the multimeric oligonucleotide is present in the composition with a purity of 85% or more. In some embodiments, the multimeric oligonucleotide is present in the composition with a purity of 90% or more. In some embodiments, the multimeric oligonucleotide is present in the composition with a purity of 95% or more. In some embodiments, the multimeric oligonucleotide is present in the composition with a purity of 99% or more.

In another aspect, the disclosure provides a pharmaceutical composition containing the multimeric oligonucleotide or composition of siRNA molecules of any of the foregoing aspects or embodiments of the disclosure and a pharmaceutically acceptable excipient, carrier, or diluent.

In a further aspect, the disclosure provides a method of delivering an siRNA molecule to the central nervous system (CNS) of a subject by administering the multimeric oligonucleotide, composition of siRNA molecules, or pharmaceutical composition of any of the foregoing aspects or embodiments of the disclosure to the CNS of the subject.

In some embodiments, the multimeric oligonucleotide, composition of siRNA molecules, or pharmaceutical composition is administered to the subject by way of intrastriatal, intracerebroventricular, or intrathecal injection.

In some embodiments, the delivering of the multimeric oligonucleotide, composition of siRNA molecules, or pharmaceutical composition to the CNS of the subject results in gene silencing of a target gene in the subject.

In some embodiments, the target gene is an overactive disease driver. In some embodiments, the target gene is a negative regulator of a gene with reduced expression that is associated with a disease state in the subject. In some embodiments, the target gene is a positive regulator of a gene with increased expression that is associated with a disease state in the subject. In some embodiments, the target gene is a splice isoform of the target gene, wherein the splice isoform reduces expression of the target gene.

In some embodiments, the gene silencing treats a disease state in the subject,

In some embodiments, the subject is a human.

In another aspect, the disclosure provides a kit containing the multimeric oligonucleotide, composition of siRNA molecules, or pharmaceutical composition of any of the foregoing aspects or embodiments of the disclosure, and a package insert, wherein the package insert instructs a user of the kit to perform a method of any of the foregoing aspects or embodiments of the disclosure.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains a hydroxyl group or modified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “multimeric oligonucleotide” refers to a composition containing 2 or more oligonucleotides (e.g., siRNA) that are contained within the same composition by way of, for example, a covalent linker or a noncovalent interaction. The noncovalent interaction may be, for example, a host-guest interaction.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently or noncovalently bound to one another. As an example, branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker. Branched siRNA molecules may have siRNA molecules covalently bonded to each other by way of a linker. Alternatively, multiple siRNA molecules may be joined by way of a noncovalent interaction.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Nonlimiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

It is understood that certain internucleotide linkages provided herein, including, e.g., phosphodiester and phosphorothioate, comprise a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

100 multiplied by (the fraction X/Y)

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of a target gene, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a gene that negatively regulates (e.g., reduces or inhibits) the expression and/or activity of another gene or set of genes (e.g., dysregulated gene or dysregulated gene pathway).

The term “positive regulator,” as used herein, refers to a gene that positively regulates (e.g., increases or saturates) the expression and/or activity of another gene or set of genes (e.g., dysregulated gene or dysregulated gene pathway).

As used herein, the term “LNA” or “locked nucleic acid” refers to a modified nucleotide in which the 2′ oxygen and 4′ carbon of the nucleotide are connected.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula CEC). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C═C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl-CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the terms “macrocyclic ring” and “macrocycle” are used interchangeably to refer to a molecule or ion that contains a ring of 12 or more atoms. Exemplary macrocyclic rings include crown ethers, calixarenes, porphyrins, and cyclodextrins.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that is suffering from, or is at risk of, a disease, disorder, or condition, as determined by a qualified professional (e.g., a doctor or a nurse practitioner) with or without known in the art laboratory test(s) of sample(s) from the subject.

As used herein, the term “reference subject” refers to a healthy control subject of the same or similar, e.g., age, sex, geographical region, and/or education level as a subject treated with a composition of the disclosure. A healthy reference subject is one that does not suffer from a disease associated with expression of a dysregulated gene or a dysregulated gene pathway. Moreover, a healthy reference subject is one that does not suffer from a disease associated with altered (e.g., increased or decreased) expression and/or activity of a gene.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of a disease. For example, clinical benefits in the context of a subject administered an siRNA molecule or siRNA composition of the disclosure include, without limitation, a reduction in the duration and/or frequency of symptoms of the disease experienced by the subject, and/or; a reduction in disease-associated phenotypes, and/or; a reduction in wild type transcripts, mutant transcripts, variant transcripts, or overexpressed transcripts, and/or splice isoforms of transcripts of a target gene.

As used herein, the term “noncovalent interaction” refers to an interaction between two atoms or groups of atoms that does not involve the sharing of electrons. A noncovalent interaction may be between atoms or groups of atoms in the same molecule or between atoms or groups of atoms in different molecules. In the context of the disclosure, two or more oligonucleotides (e.g., siRNA) may be joined together by way of a noncovalent interaction, either between two or more nucleic acids or by way of a host-guest interaction. Exemplary noncovalent interactions include electrostatic interactions, Lewis acid-base interactions, hydrogen-bonding interactions, hydrophobic interactions, Van der Waals interactions, a π-effect (e.g., π stacking, cation-π interactions, anion-π interactions, metal-π interactions, or polar-π interactions).

It will be appreciated by one of skill in the art that two or more molecules may be noncovalently bound to each other by one of the noncovalent interactions described herein or by two or more of the interactions described herein, and the multimeric oligonucleotides of the present disclosure are therefore not limited to those containing only a single type of noncovalent interaction. For example, in a nucleic acid hybridization event, the two strands form a duplex that is stabilized by both hydrogen bonds (between hydrogen atoms in one nucleobase and nitrogen or oxygen atoms in the other) and by T-stacking among the aromatic nucleobases. As a further example, a protein-ligand interaction, which in and of itself may be considered a noncovalent interaction, may involve two or more noncovalent interactions. For example, a small molecule ligand may contain heteroatoms that can undergo hydrogen bonding interactions with hydrogen bond donors/acceptors in the protein as well as nonpolar aromatic groups that can undergo π-stacking and/or Van der Waals interactions with nonpolar residues in the protein.

As used herein, a “noncovalent binding moiety” refers to a substituent in one compound (or in one location of a compound) that is capable of undergoing a noncovalent interaction with another substituent on another compound (or in another location of the same compound). A noncovalent binding moiety is used in the context of this disclosure to join 2 or more siRNA molecules. For example, a biotin group is a noncovalent binding moiety that may bind to avidin (or an analog thereof). A nucleic acid may also be considered a noncovalent binding moiety, as it is capable of forming a noncovalent interaction with another nucleic acid by way of nucleic acid hybridization.

As used herein, the term “host-guest” or “guest-host” refers to a complex composed by two or more molecules that are held together by noncovalent interactions. A “host-guest complex” is a supramolecule in which one or more guest molecules is bound to one or more host molecules by way of a noncovalent interaction. Exemplary noncovalent interactions of host-guest molecules include, but are not limited to, electrostatic interactions, hydrogen bonds, van der Waal's interactions, and hydrophobic interactions.

As used herein, the term “electrostatic interaction” refers to an attractive or repulsive interaction between charged molecules.

As used herein, the term “Lewis acid” refers to a chemical species that contains an empty orbital that is capable of accepting an electron pair. A “Lewis base” is a chemical species that has an electron pair capable of being donated to an empty orbital of another chemical species. A “Lewis acid-base interaction” as used herein refers specifically to the formation of a metal complex between a metal cation and a Lewis base.

As used herein, the term “hydrogen bond” or “hydrogen bonding” refers to the interaction between a hydrogen atom that is bound to an electronegative atom (e.g., nitrogen, oxygen, fluorine, or sulfur), which is referred to as a hydrogen bond donor, and another electronegative atom, (e.g., nitrogen, oxygen, fluorine, or sulfur) which is referred to as a hydrogen bond acceptor. A hydrogen bond may be intramolecular or intermolecular.

As used herein, the term “hydrophobic interaction” or “hydrophobic effect” refers to the effect of nonpolar substances aggregating in an aqueous solution to exclude water molecules.

As used herein, the term “van der Waals force” or “van der Waals interaction” refers distance-dependent attractive or repulsive forces between two or more atoms or molecules other than those caused by ionic or covalent bonding. Examples include dipole-dipole interactions, dipole-induce dipole interactions, and London dispersion interactions.

As used herein, the term “π-effect” includes π-π interactions, cation-π, and anion-IT interactions that are caused by the distortion of electron density within a IT system of an aromatic ring. This distortion can lead to a noncovalent interaction with another π-system, an anion, or a cation.

As used herein, the term “protein-ligand interaction” refers to a reversible interaction between a protein and another molecule, such as a small molecule, a peptide, or a nucleic acid. The interaction may be formed by any of the noncovalent interactions of the disclosure.

As used herein, the term “antibody” (Ab) refers to an immunoglobulin molecule that specifically binds to, or is immunologically reactive with, a particular antigen, and includes polyclonal, monoclonal, genetically engineered and otherwise modified forms of antibodies, including but not limited to chimeric antibodies, humanized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, recombinant IgG (rIgG) fragments, and scFv fragments. Moreover, unless otherwise indicated, the term “monoclonal antibody” (mAb) is meant to include both intact molecules, as well as antibody fragments (such as, for example, Fab and F(ab′)₂ fragments) that are capable of specifically binding to a target protein. Fab and F(ab′)₂ fragments lack the Fc fragment of an intact antibody, clear more rapidly from the circulation of the animal, and may have less non-specific tissue binding than an intact antibody (see Wahl et al., J. Nucl. Med. 24:316, 1983; incorporated herein by reference).

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

### BRIEF DESCRIPTION OF THE FIGURES

FIG. 1A shows an exemplary structure of an siRNA “monomer” where one or more double stranded siRNA molecule is covalently attached, by way of a linker, to a single stranded nucleic acid.

FIG. 1B depicts how two or more siRNA molecules may be joined to form di-, tri-, and tetra-branched siRNA molecules by way of the hybridization/annealing of the nucleic acids that are contained within each monomer.

FIG. 1C shows exemplary noncovalently branched, multimeric oligonucleotides that contain one or mismatches denoted by “x.” The mismatches may be contained within the nucleic acids used to form the noncovalent interaction, within the siRNA molecule, or both.

FIG. 1D shows exemplary noncovalently branched, multimeric oligonucleotides that contain overhangs in either the nucleic acids that hybridize or the siRNA molecules.

In FIGS. 1A-D, dashed lines indicate linkers, bold lines indicate the nucleic acids that hybridize, and regular lines indicate the siRNA molecules. Horizontal lines that are connected by a vertical line indicated a matched base pair between the two nucleic acids. Horizontal lines with a hash mark that does not contain a vertical line above or below it indicates a single stranded portion that is not paired with another nucleic acid. An “x” indicates a base pair that is a mismatched base pair within a hybridized nucleic acid.

FIG. 2A shows a general synthetic scheme for the preparation of a noncovalently branched tetrameric siRNA that is branched by a biotin/streptavidin interaction.

FIG. 2B shows a general synthetic scheme for the preparation of a noncovalently branched dimeric siRNA that is branched by a locked nucleic acid hybridization interaction.

FIG. 3A shows gel electrophoresis analysis in the preparation of a noncovalently branched dimeric siRNA that is branched by a locked nucleic acid hybridization interaction.

FIG. 3B is an anion exchange HPLC trace of a noncovalently branched dimeric siRNA that is branched by a locked nucleic acid hybridization interaction.

FIG. 3C is an anion exchange HPLC trace of a noncovalently branched tetrameric siRNA that is branched by a biotin/streptavidin interaction.

FIG. 3D is a size exclusion chromatogram analyzing a biotin/streptavidin conjugated siRNA after mixing biotinylated siRNA and streptavidin together in a 4:1 ratio of biotinylated siRNA to streptavidin.

FIG. 3E is a size exclusion chromatogram showing conversion from the monomeric biotinylated siRNA to the tetrameric biotin/streptavidin conjugated siRNA after titration with additional streptavidin to the mixture shown in FIG. 3D.

FIG. 4 shows the in vitro efficacy of branched siRNA molecules. The siRNA molecules were a) a covalently branched dimeric siRNA of Formula XVII, b) a noncovalently branched tetrameric siRNA that is branched by a biotin/streptavidin interaction, and c) a noncovalently branched dimeric siRNA that is branched by a locked nucleic acid hybridization interaction. Di-branched siRNAs were reverse transfected into Hela cells at the indicated concentrations for 72 hrs

FIG. 5 shows the in vivo efficacy of branched siRNA molecules of the disclosure in female FVB/NJ mice that were treated with the siRNA molecules by way of a bilateral intracerebroventricular injection. The graph indicates the knockdown of HPRT1 mRNA in four different brain regions (frontal cortex, motor cortex, striatum, and hippocampus) using both covalently and noncovalently branched siRNA molecules.

### DETAILED DESCRIPTION

The present invention provides multimeric oligonucleotides containing short-interfering RNA (SiRNA) molecules, including single- and double-stranded short interfering RNA (ds-siRNA), and methods for their use in treating a patient in need of gene silencing (e.g., a patient having dysregulated gene expression, such as a patient with, e.g., Alzheimer's disease, amyotrophic lateral sclerosis, Parkinson's disease, frontotemporal dementia, Huntington's disease, multiple sclerosis, or progressive supranuclear palsy). siRNA molecules are capable of mediating RNA interference (RNAi) by degrading mRNA with a complementary nucleotide sequence, thus reducing, or altogether preventing, the translation of the target gene. The siRNA molecules of the disclosure may be in the form of multimeric oligonucleotides formed by way of noncovalent interactions, e.g., host-guest interactions. The siRNA molecules described herein may employ a variety of chemical modifications. For example, the siRNA molecules described herein may include specific patterns of chemical modifications (e.g., 2′ ribose modifications or internucleoside linkage modifications) to improve resistance against nuclease enzymes, toxicity profile, and physicochemical properties (e.g., thermostability).

The siRNA molecules of the disclosure may feature an antisense strand having a nucleic acid sequence that is complementary to a region of an mRNA transcript in a target gene. The degree of complementarity of the antisense strand to the region of the target mRNA transcript may be sufficient for the antisense strand to anneal over the full length of the region of the mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the target mRNA transcript.

siRNA Structure

The siRNA molecules of the disclosure may be in the form of a single-stranded (ss) or double-stranded (ds) RNA structure. In some embodiments, the siRNA molecules may be di-branched, tri-branched, or tetra-branched molecules. The siRNA molecules may be in the form of a multimeric oligonucleotide formed by way of a noncovalent host-guest interaction. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently or noncovalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates, phosphodiesters, etc.). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

A-B-(A′)ⱼ-C-P²-D-P¹-(C′-P¹)ₖ-C′ Formula I;

wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-B-S-A Formula A1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

A-B-(A′)ⱼ-C-P²-D-P¹-(C-P¹)ₖ-C′ Formula II;

wherein A is represented by the formula C-P¹-D-P¹; each A′ is represented by the formula C-P²-D-P²; B is represented by the formula C-P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

A-S-B-S-A-O-B-O-B-O-B-O-A-O-B-O-A-O-B-O-A-O-B-O-A-O-B-O-A-O-B-S-A-S-A-S-A-S-A-S-A Formula A2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

E-(A′)ₘ-F Formula III;

wherein E is represented by the formula (C-P¹)₂; F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

A-S-A-S-A-O-B-O-A-O-B-O-A-O-B-O-A-O-B-O-A-O-A-O-A-O-B-S-A-S-A Formula S1;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

A-S-A-S-A-O-B-O-A-O-B-O-A-O-B-O-A-O-B-O-A-O-A-O-A-O-B-O-A-O-A Formula S2;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

A-S-A-S-A-O-B-O-A-O-B-O-A-O-B-O-A-O-B-O-A-O-A-O-A-O-B-S-A-S-B Formula S3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

A-S-A-S-A-O-B-O-A-O-B-O-A-O-B-O-A-O-B-O-A-O-A-O-A-O-B-O-A-O-B Formula S4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

A-(A′)ⱼ-C-P²-B-(C-P¹)ₖ-C′ Formula IV;

wherein A is represented by the formula C-P¹-D-P¹; each A′ is represented by the formula C-P²-D-P²; B is represented by the formula D-P¹-C-P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

A-S-B-S-A-O-B-O-A-O-B-O-A-O-B-O-A-O-B-O-A-O-B-O-A-O-B-O-A-O-B-S-A-S-B-S-A-S-A-S-A Formula A3;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

E-(A′)ₘ-C-P²-F Formula V;

wherein E is represented by the formula (C-P¹)₂; F is represented by the formula D-P¹-C-P¹-C, D-P²-C-P²—C, D-P¹-C-P¹-D, or D-P²-C-P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

A-S-A-S-A-O-B-O-A-O-B-O-A-O-B-O-A-O-B-O-A-O-B-O-A-O-B-S-A-S-A Formula S5;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

A-S-A-S-A-O-B-O-A-O-B-O-A-O-B-O-A-O-B-O-A-O-B-O-A-O-B-O-A-O-A Formula S6;

wherein A represents a 2′-O-Me ribonucleoside, B represents a 2′-F ribonucleoside, O represents a phosphodiester internucleoside linkage, and S represents a phosphorothioate internucleoside linkage.
In some embodiments of the disclosure, the sense strand includes a structure represented by Formula S7, wherein Formula S7 is, in the 5′-to-3′ direction:

A-S-A-S-A-O-B-O-A-O-B-O-A-O-B-O-A-O-B-O-A-O-B-O-A-O-B-S-A-S-B Formula S7;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

A-S-A-S-A-O-B-O-A-O-B-O-A-O-B-O-A-O-B-O-A-O-B-O-A-O-B-O-A-O-B Formula S8;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

A-Bⱼ-E-Bₖ-E-F-GI-D-P¹-C′ Formula VI;

wherein A is represented by the formula C-P¹-D-P¹; each B is represented by the formula C-P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²-C-P²; F is represented by the formula D-P¹-C-P¹; each G is represented by the formula C-P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and I is an integer from 1 to 7 (e.g., 1, 2, 3, 4, 5, 6, or 7). In some embodiments, j is 3. In some embodiments, k is 6. In some embodiments, I is 2. In some embodiments, j is 3, k is 6, and I is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

A-S-B-S-A-O-A-O-A-O-B-O-A-O-A-O-A-O-A-O-A-O-A-O-A-O-B-O-A-O-B-S-A-S-A-S-A-S-B-S-A Formula A4;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

H-Bₘ—Iₙ-A′-Bₒ—H-C Formula VII;

wherein A′ is represented by the formula C-P²-D-P²; each H is represented by the formula (C-P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

A-S-A-S-A-O-A-O-A-O-B-O-B-O-B-O-A-O-B-O-A-O-A-O-A-O-A-S-A-S-A Formula S9;

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

Z-((A-P-)ₙ(B-P-)ₘ)_{q}; Formula VIII

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

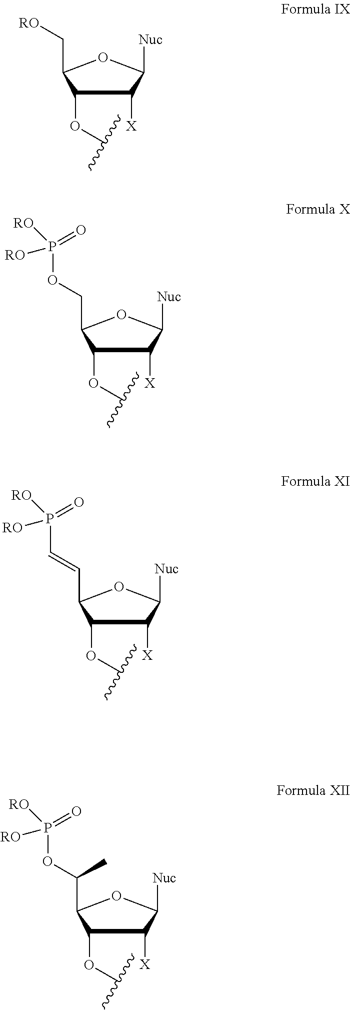

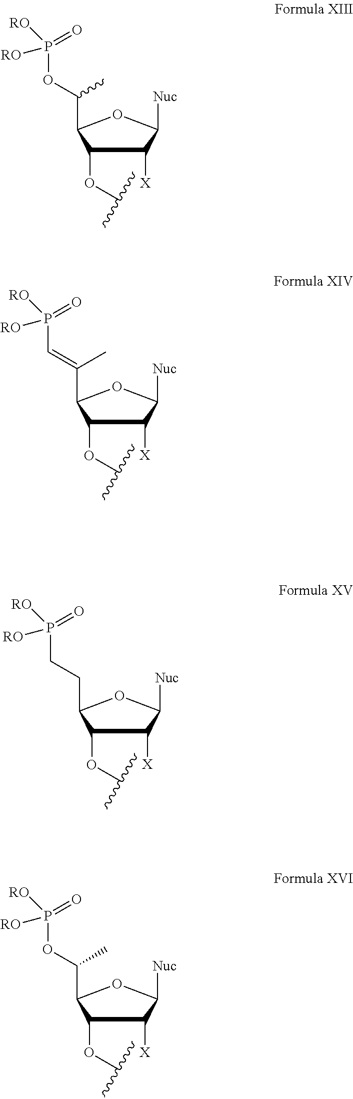

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

siRNA Branching

The present disclosure includes branched siRNA molecules that are joined by way of noncovalent interactions. The siRNA molecules may also be joined by a covalent linkage. Alternatively, the siRNA molecules may be joined by way of a combination of noncovalent and covalent linkages.

Noncovalently Linked siRNA

The branched siRNA molecules of the disclosure may contain 2, 3, 4, 5, 6, 7, or 8 siRNA molecules that are noncovalently attached. The siRNA molecules may be joined by way of a noncovalent binding moiety allowing the siRNA molecule to noncovalently bind to at least one other siRNA molecule, where each siRNA molecule is attached to the noncovalent binding moiety by way of a linker.

For example, each siRNA may be covalently attached to a nucleic acid. One or more other siRNA molecules may be attached to a nucleic acid that is complementary to the first nucleic acid, allowing for the siRNA molecules to combine by way of a nucleic acid hybridization event. In some embodiments, at least one noncovalent binding moiety is a nucleic acid or a nucleic acid analog. In some embodiments, the noncovalent interaction is nucleic acid hybridization. The nucleic acids used for hybridization may be DNA or RNA, or a nucleic acid analog such as a PNA, an LNA, a GNA, a TNA, an HNA, or a morpholino oligomer.

The noncovalent attachment may be by way of a host-guest interaction, wherein 2 or more siRNA molecules each contain a guest moiety that binds to a host molecule. For example, the siRNA compositions of the disclosure may have the following structure:

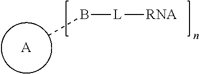

wherein A is a host molecule;
each B is, independently, a guest moiety that noncovalently binds to the host molecule;
each --- is a noncovalent interaction between the host molecule and the guest moiety;
each L is, independently, absent or a linker;
each RNA is, independently, an siRNA molecule; and
n is an integer from 2-8.

In some embodiments, the multimeric oligonucleotide is of the following structure:

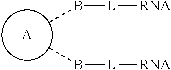

In some embodiments, the multimeric oligonucleotide is of the following structure:

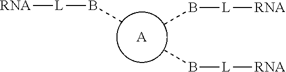

In some embodiments, the multimeric oligonucleotide is of the following structure:

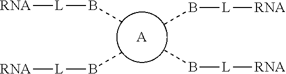

Covalently Linked siRNA

Alternatively, the siRNA molecules disclosed herein may be covalently linked branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. The branched siRNA molecules of the disclosure may contain 2, 3, 4, 5, 6, 7, or 8 siRNA molecules that are covalently attached by way of a linker. Some exemplary embodiments are listed in Table 1.

TABLE 1
Branched siRNA strctures

| Di-branched | Tri-branched | Tetra-branched |
|---|---|---|
| RNA-L-RNA Formula XVII | 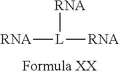 | 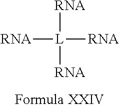 |
| 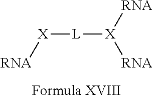 | 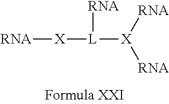 | 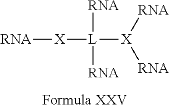 |
| 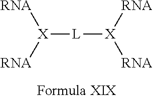 | 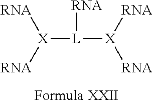 | 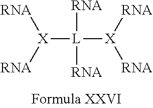 |
| | 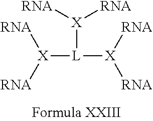 | 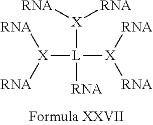 |
| | | 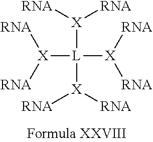 |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

In addition to the noncovalent interactions disclosed herein, multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

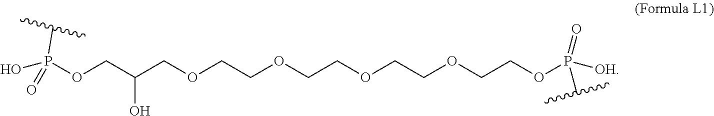 [SMILES: *P(=O)(O)OCCOCCOCCOCCOCC(O)COP(*)(=O)O]

In some embodiments, the linker has a structure of Formula L2:

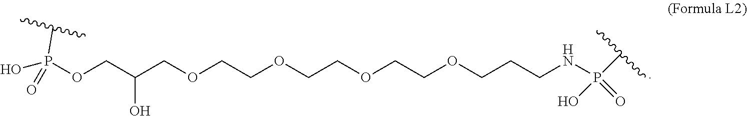 [SMILES: *P(=O)(O)NCCCOCCOCCOCCOCC(O)COP(*)(=O)O]

In some embodiments, the linker has a structure of Formula L3:

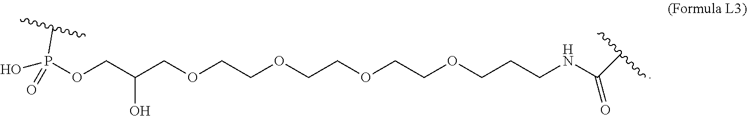 [SMILES: *C(=O)NCCCOCCOCCOCCOCC(O)COP(*)(=O)O]

In some embodiments, the linker has a structure of Formula L4:

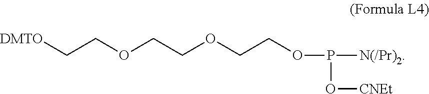

In some embodiments, the linker has a structure of Formula L5:

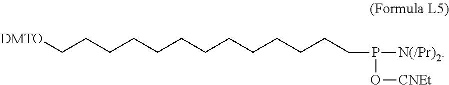

In some embodiments, the linker has a structure of Formula L6:

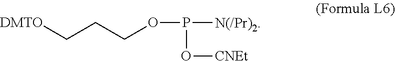

In some embodiments, the linker has a structure of Formula L7, as is shown below:

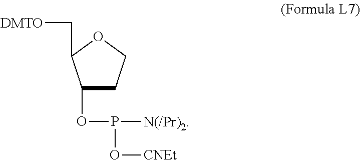

In some embodiments, the linker has a structure of Formula L8:

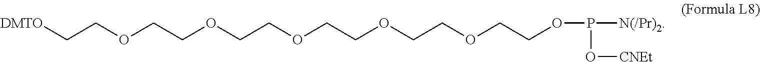

In some embodiments, the linker has a structure of Formula L9:

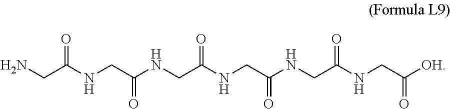 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Noncovalent Interactions

The siRNA molecules of the disclosure may be in the form of noncovalently branched, multimeric oligonucleotides. The multimeric oligonucleotides may contain from 2 to 8 nucleotides that each contain a noncovalent binding moiety. The noncovalent binding moiety may be a guest moiety that can bind to a host molecule to form the multimeric oligonucleotide.

### Nucleic Acid Hybridization

The noncovalent interaction may involve hybridization of complementary nucleic acids. The hybridization may include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The nucleic acids involved in the noncovalent interaction may be deoxyribonucleic acids or ribonucleic acids. They may be a modified nucleic acid, such as a 2′-sugar modified nucleic acid (e.g., 2′-fluoro, 2′-methoxy, 2′-methoxyethoxy). They may also contain modified internucleoside linkages, such as phosphorothioate linkages that may or may not be stereochemically enriched.

The nucleic acids involved in the noncovalent interaction may be a nucleic acid analog. For example, the nucleic acid analog may be a peptide nucleic acid (PNA), a locked nucleic acid (LNA), a glycol nucleic acid (GNA), a threose nucleic acid (TNA), a hexitol nucleic acid (HNA), or a morpholino oligomer.

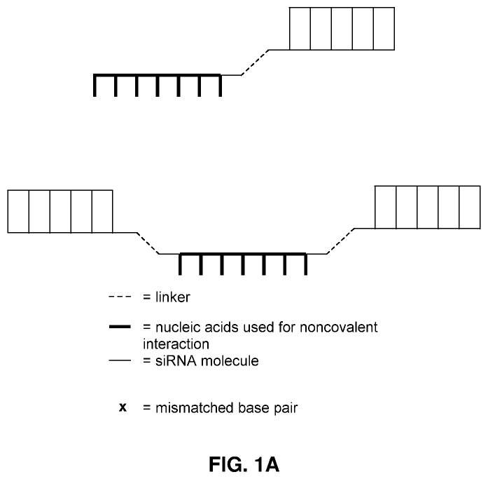

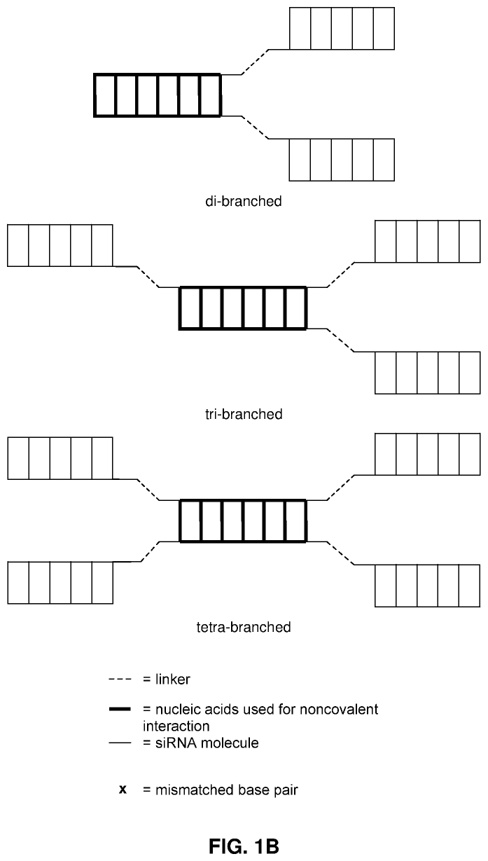

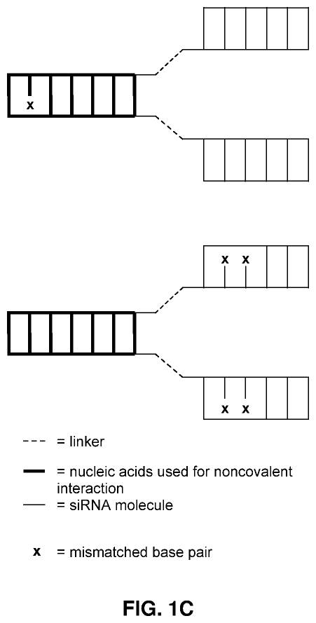

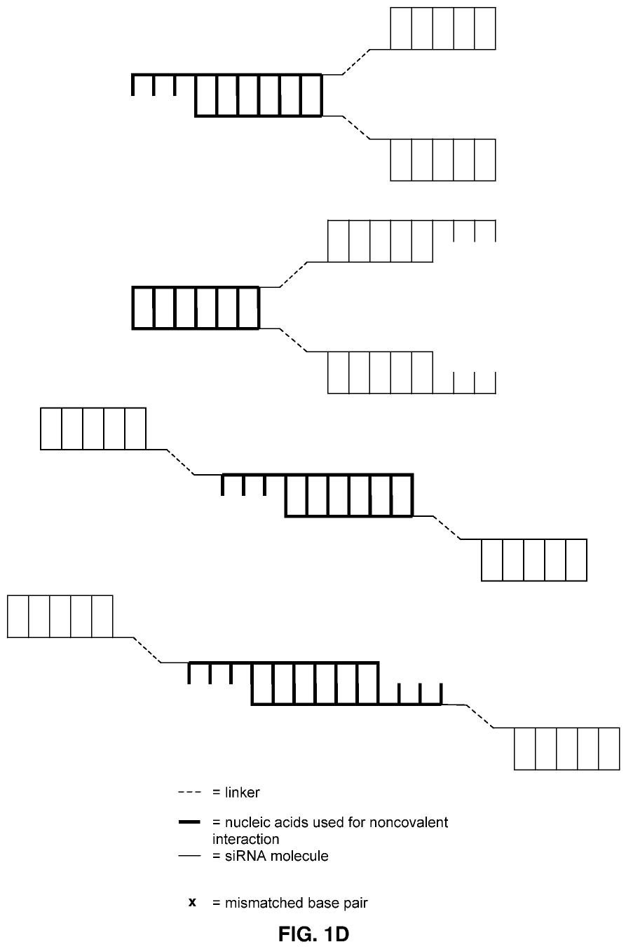

The noncovalent joining of multiple siRNA molecules begins with one or more double stranded siRNA molecules (ds-siRNA) attached to a single stranded nucleic acid by way of a linker (FIG. 1A). One or more other ds-siRNA attached to a complementary single stranded nucleic acid will allow for the nucleic acids to join by way of hybridization of the single stranded nucleic acids to form a stable duplex. Each nucleic acid may be linked to a single siRNA to form a di-branched siRNA. One or both of the nucleic acids may be linked to two or more siRNA molecules to form, for example, a tri-branched or tetra-branched SIRNA (FIG. 1B). It should be noted that, as stated above, nucleic acids need not be 100% complementary in order to anneal/hybridize, and there may be one or more mismatches in the nucleic acids that hybridize and/or the siRNA molecules (FIG. 1C). Furthermore, the nucleic acids used in the hybridization and/or the siRNA molecules may be the same length or may be different lengths and contain overhangs. The overhang may be at the 5′-end of the strand, the 3′-end of the strand, or both (FIG. 1D). FIG. 1A-FIG. 1D are meant to be illustrative of the features that may be contained in the multimeric oligonucleotides only and are not drawn to scale and are not intended to be limiting. The multimeric oligonucleotide compositions disclosed herein may none of the features described above, or they may have, without limitation, any combination of one or more of the features described in the preceding paragraph (e.g., they may contain mismatches as well as contain overhangs) as well as other features not described herein but that are known and understood by one of skill in the art.

In some embodiments, the multimeric oligonucleotide has the following structure:

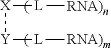

wherein X and Y are each, independently, a nucleic acid or a nucleic acid analog;
each of m and n is, independently, 1, 2, 3, or 4;
each L is, independently, absent or a linker;
each RNA is, independently, an siRNA molecule;
wherein X and Y have complementarity sufficient to hybridize to one another; and
--- represents a hybridization interaction between X and Y.

In some embodiments, the sum of m and n is 2. In some embodiments, the sum of m and n is 3. In some embodiments, the sum of m and n is 4. In some embodiments, the sum of m and n is 5. In some embodiments, the sum of m and n is 6. In some embodiments, the sum of m and n is 7. In some embodiments, the sum of m and n is 8.

In some embodiments, the sum of m and n is 2 and the multimeric oligonucleotide contains a first SIRNA molecule and a second siRNA molecule. In some embodiments, the first siRNA molecule is attached the to the 5′ end of nucleic acid X and the second siRNA molecule is attached to the 5′ end of nucleic acid Y. In some embodiments, the first siRNA molecule is attached the to the 5′ end of nucleic acid X and the second siRNA molecule is attached to the 3′ end of nucleic acid Y. In some embodiments, the first siRNA molecule is attached the to the 3′ end of nucleic acid X and the second siRNA molecule is attached to the 3′ end of nucleic acid Y. In some embodiments, the first siRNA molecule is attached the to the 3′ end of nucleic acid X and the second siRNA molecule is attached to the 5′ end of nucleic acid Y.

In some embodiments, the sum of m and n is 3 and the multimeric oligonucleotide contains a first siRNA molecule, a second siRNA molecule, and a third siRNA molecule. In some embodiments, the first SiRNA molecule is attached the to the 5′ end of nucleic acid X, the second siRNA molecule is attached to the 3′ end of nucleic acid X, and the third siRNA molecule is attached to the 3′ end of nucleic acid Y. In some embodiments, the sum of m and n is 3 and the multimeric oligonucleotide contains a first siRNA molecule, a second siRNA molecule, and a third siRNA molecule. In some embodiments, the first siRNA molecule is attached the to the 5′ end of nucleic acid X, the second siRNA molecule is attached to the 3′ end of nucleic acid X, and the third siRNA molecule is attached to the 5′ end of nucleic acid Y.

### Cyclodextrin Interactions

The host-guest interaction may be a cyclodextrin-adamantane interaction. The interaction may include different adamantane to cyclodextrin ratios, for example, as those described in Wang et. Al., Molecules, 26:2412, 2021, the disclosure of which is incorporated herein by reference. In some embodiments of the disclosure, the host molecule is a macrocyclic ring. The macrocyclic ring is a molecule or ion that contains a ring of 12 or more atoms. For example, the macrocyclic ring may be a cyclodextrin. In some embodiments, the cyclodextrin has any of the following structures:

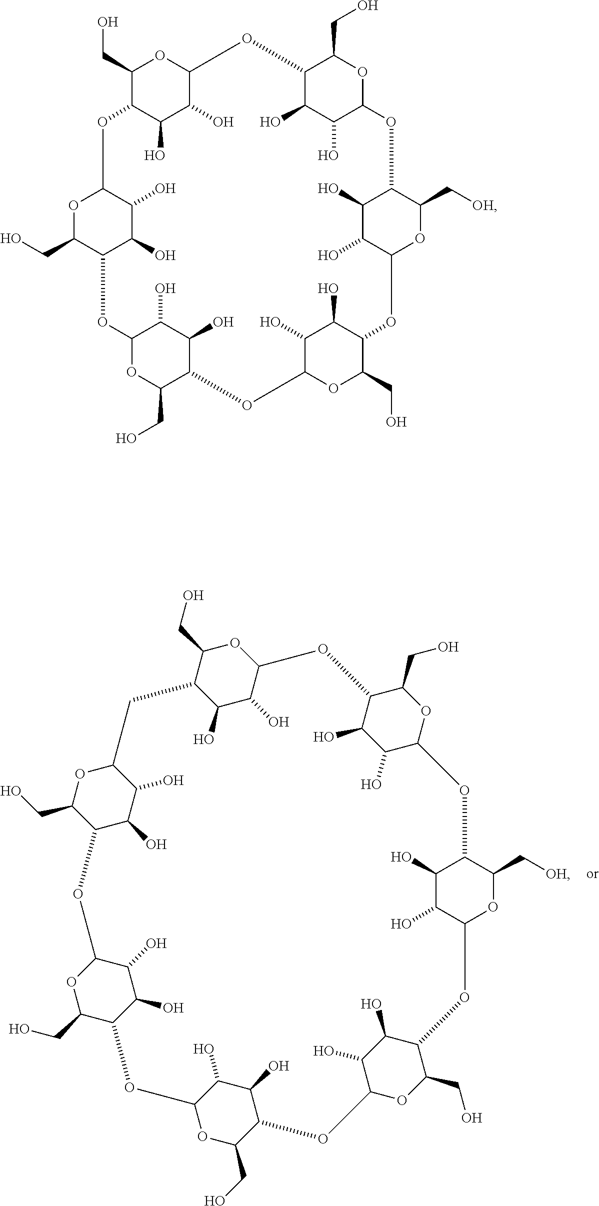

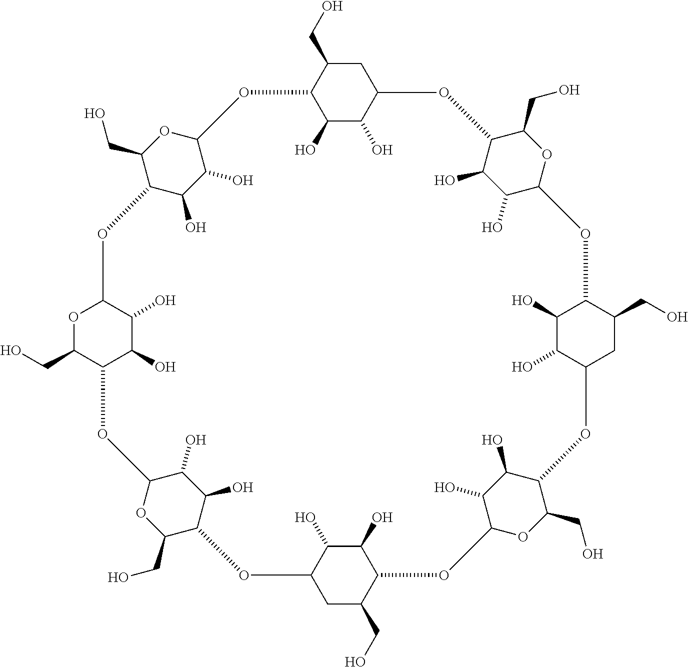

In some embodiments, 1 or more (e.g., 1 or more, 3 or more, 5 or more, 7 or more, 9 or more, 11 or more, 13 or more, or 15 or more) —OH groups within the cyclodextrin may be substituted with —OR^{A}, wherein R^{A} is optionally substituted C₁₋₆ alkyl. Other exemplary cyclodextrin analogs are described in U.S. Pat. Nos. 8,114,438; 10,662,260; and in Saenger et. Al., Chem. Rev 98:1787, 1998; Bodine et. Al., J. Am. Chem. Soc., 126:1638, 2004; Lepage et. Al., J. Org. Chem., 80:10719, 2015; Li et. Al., J. Am. Chem. Soc., 133:1987, 2011; Crini Chem. Rev 114:10940, 2014; Schönbeck et. Al., Langmuir, 27:5832, 2011; Li, et. Al., J. Org. Chem., 75:6673, 2010; Alcalde et. Al., J. Phys. Chem. B, 110:13399, 2006; and Schneider et. Al., ChemistrySelect, 5:10765, 2020, the disclosure of each of which are incorporated herein by reference.

In some embodiments, each guest molecule is, independently:

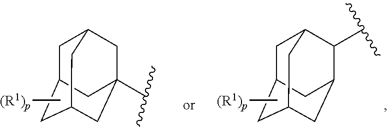

wherein each R¹ is, independently, hydroxyl, optionally substituted amino, optionally substituted alkoxy, optionally substituted C₁₋₆ alkyl, or —CO₂H; and
p is 0, 1, 2, 3, 4, or 5.

In some embodiments, p is 0. In some embodiments, p is 1. In some embodiments, p is 2.

In some embodiments, R¹ is hydroxyl. In some embodiments, each B is, independently:

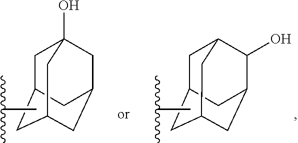

In some embodiments, each B is, independently:

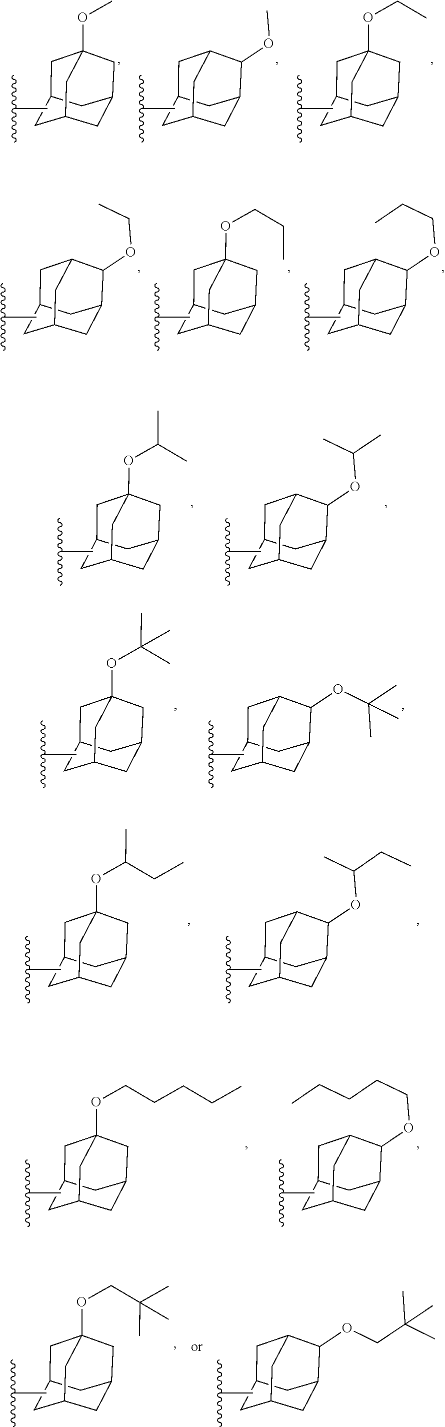

wherein

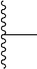

represents a point of attachment to L and is substituted at any hydrogen atom in B.

In some embodiments, R¹ is optionally substituted amino. In some embodiments, R¹ is —NH₂ In some embodiments, each B is:

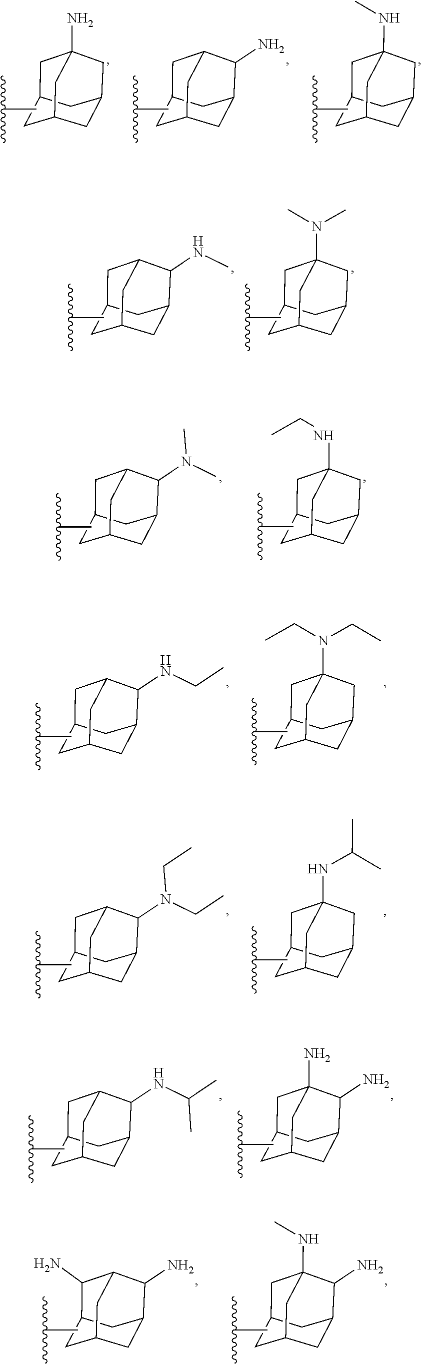

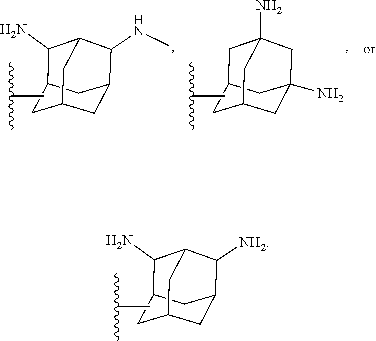

### Wherein

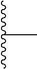

represents a point of attachment to L and is substituted at any hydrogen atom in B.

In some embodiments, R¹ is —CO₂H. In some embodiments, each guest molecule is:

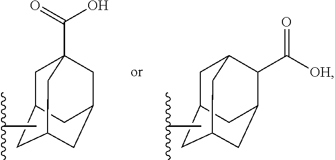

wherein

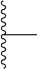

represents a point of attachment to L and is substituted at any hydrogen atom in B.

In some embodiments, each quest molecule is:

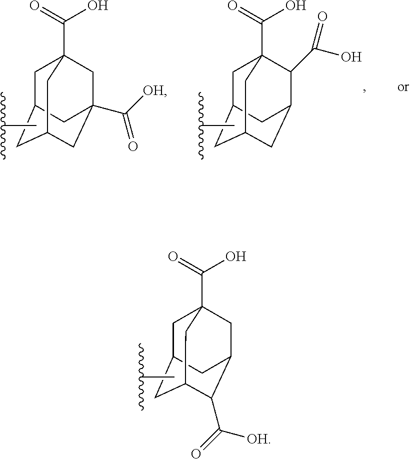

### Wherein

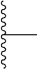

represents a point of attachment to L and is substituted at any hydrogen atom in B.

In some embodiments, R¹ is optionally substituted C₁₋₆ alkyl. In some embodiments, each guest molecule is:

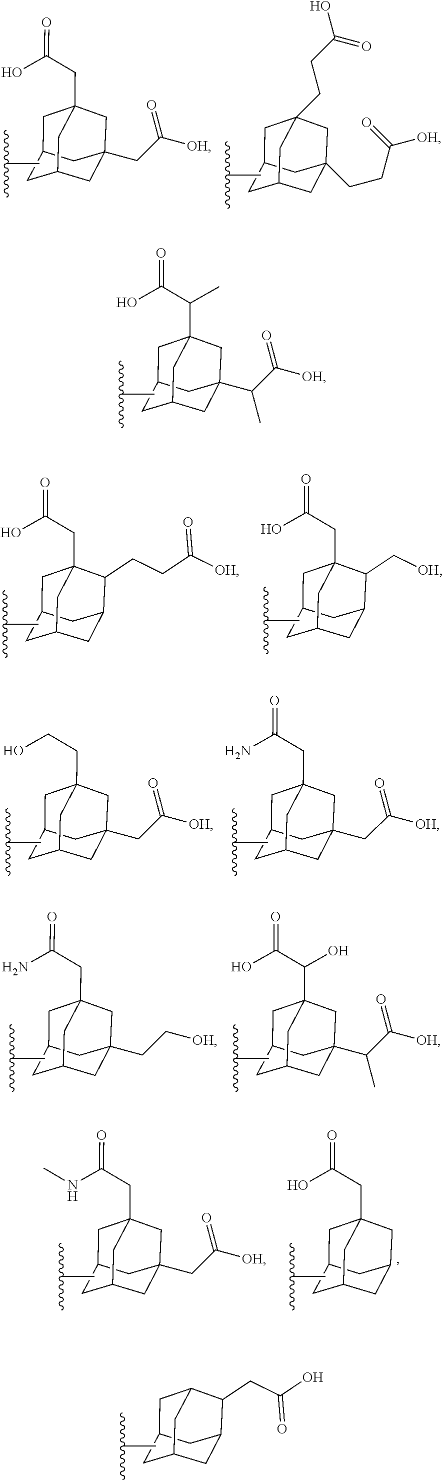

wherein

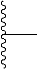

represents a point of attachment to L and is substituted at any hydrogen atom in B.

Cyclodextrins are also known to effectively bind other hydrophobic molecules that would be a suitable guest partner for forming a multimeric oligonucleotide. Accordingly, in some embodiments, B is a sterol (e.g., cholesterol) analog.

### Transferrin-Transferrin Binding Protein Interactions

Multimeric oligonucleotides of the disclosure may be formed by way of transferrin-transferrin binding protein interactions. An siRNA molecule may be covalently attached to transferrin (Tf) or an analog thereof which binds to a transferrin binding protein (Tbp) or an analog thereof. Tf-Tbp interactions are discussed, for example, in U.S. Patent Application No. 2004/0258695 and 2006/0034854, the disclosure of each of which are incorporated herein by reference. The transferrin binding protein may be TbpA or TbpB, or an analog thereof, or an isoform thereof.

The amino acid sequence of an exemplary human transferrin protein is shown in UNIPROT accession number P02787. The transferrin used in the present disclosure may be at least 85% identical (e.g., 85%, 86%, 97%, 88%, 89%, 90%, 91%, 92%, 93%, 94%, 95%, 96%, 97%, 98%, 99%, or 100% identical) to the aforementioned amino acid sequence.

The amino acid sequence of an exemplary TbpA protein encoded by Neisseria meningitidis is shown in UNIPROT accession number Q9K0U9. The TbpA used in the present disclosure may be at least 85% identical (e.g., 85%, 86%, 97%, 88%, 89%, 90%, 91%, 92%, 93%, 94%, 95%, 96%, 97%, 98%, 99%, or 100% identical) to the aforementioned amino acid sequence.

The amino acid sequence of an exemplary TbpB protein encoded by Neisseria meningitidis is shown in UNIPROT accession number Q9K0V0. The TbpB used in the present disclosure may be at least 85% identical (e.g., 85%, 86%, 97%, 88%, 89%, 90%, 91%, 92%, 93%, 94%, 95%, 96%, 97%, 98%, 99%, or 100% identical) to the aforementioned amino acid sequence.

### Biotin-Avidin Interactions

Multimeric oligonucleotides of the disclosure may be formed by way of biotin-avidin interactions. An siRNA molecule may be covalently attached to biotin or an analog thereof which binds to avidin or an analog thereof.

In some embodiments, host molecule is avidin. In some embodiments, the host molecule is an avidin analog. For example, the avidin analog may be, streptavidin, NeutrAvidin, Bradavidin II, hoefavidin, rhizavidin, Tamavidin 2, Shwanavidin, Switchavidin, Zebavidin, or Strep-Tactin®. In some embodiments, the avidin analog is bound to a solid support.

In some embodiments, each guest molecule is, independently, a peptide. In some embodiments, each B has the peptide sequence WSHPQFEK (SEQ ID NO: 1).

In some embodiments, each guest molecule is, independently:

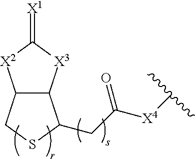

wherein
X¹, X², X³ and X⁴ are each, independently, O or NR²;
each R₂ is, independently, H or optionally substituted C₁₋₆ alkyl;
r is 0 or 1; and
s is 1, 2, 3, 4, 5, or 6.

In some embodiments, X¹ is O. In some embodiments, X¹ is NR². In some embodiments, X² is NR². In some embodiments, X³ is NR². In some embodiments, R² is H. In some embodiments, R² is optionally substituted C₁₋₆ alkyl. In some embodiments, R² is ethyl. In some embodiments, X² is O. In some embodiments, X³ is O. In some embodiments, r is 0. In some embodiments, r is 1. In some embodiments, s is 1. In some embodiments, s is 2. In some embodiments, s is 3. In some embodiments, s is 4. In some embodiments, s is 5. In some embodiments, s is 6.

The guest moiety may have any of the following structures, or a salt thereof, or a stereoisomer thereof:

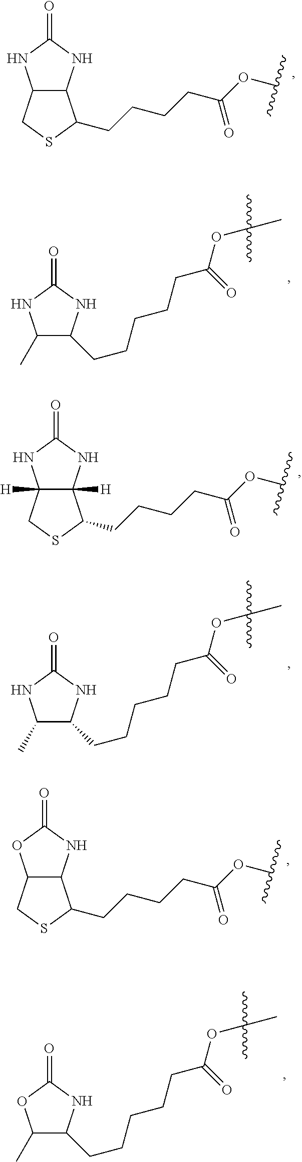 [SMILES: *OC(=O)CCCCC1SCC2NC(=O)NC21 ; *OC(=O)CCCCCC1NC(=O)NC1C ; *OC(=O)CCCC[C@@H]1SC[C@@H]2NC(=O)N[C@@H]21 ; *OC(=O)CCCCC[C@H]1NC(=O)N[C@H]1C ; *OC(=O)CCCCC1SCC2OC(=O)NC21 ; *OC(=O)CCCCCC1NC(=O)OC1C]

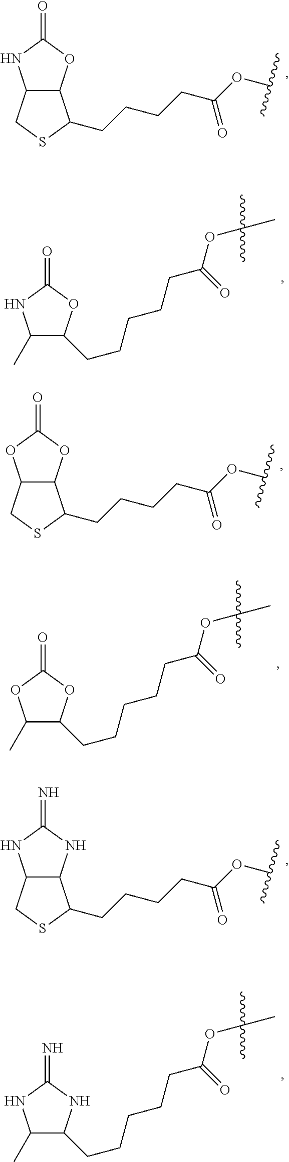

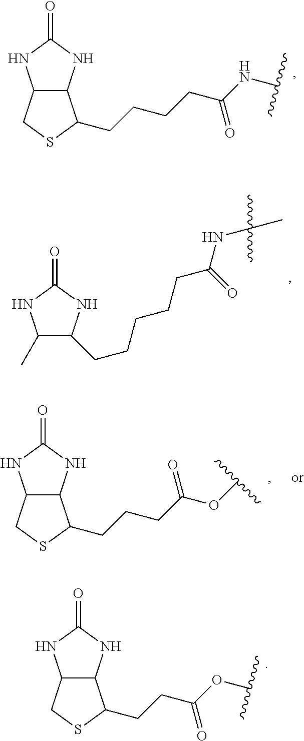 [SMILES: *NC(=O)CCCCC1SCC2NC(=O)NC21 ; *NC(=O)CCCCCC1NC(=O)NC1C ; *OC(=O)CCCC1SCC2NC(=O)NC21 ; *OC(=O)CCC1SCC2NC(=O)NC21]

### Metal-Ligand Interactions

The present disclosure also provides multimeric oligonucleotides that are joined by way of a metal-ligand interaction. For example, 2 or more siRNA molecules may each, independently, contain a guest moiety that is a ligand that coordinates to a metal. In some embodiments, the metal is iron. In some embodiments, the metal is copper, manganese, magnesium, zinc, or gadolinium. In particular embodiments, the metal is iron (III). Each ligand may be a monodentate ligand, a bidentate ligand, or a tridentate ligand. The metal-ligand complex may adopt a linear geometry, a trigonal planar geometry, a tetrahedral geometry, a square planar geometry, a trigonal bipyramidal geometry, a square pyramidal geometry, or an octahedral geometry.

Each ligand may, independently, have the following structure:

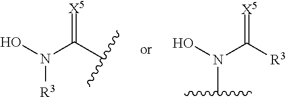

wherein
R³ is hydrogen, optionally substituted C₁-C₆ alkyl, optionally substituted C₁-C₆ alkenyl, optionally substituted C₁-C₆ alkynyl;
X⁵ is O, S, or NR⁴; and
R⁴ is H or optionally substituted C₁₋₆ alkyl.

In particular embodiments, each ligand may, independently, have any of the following structures:

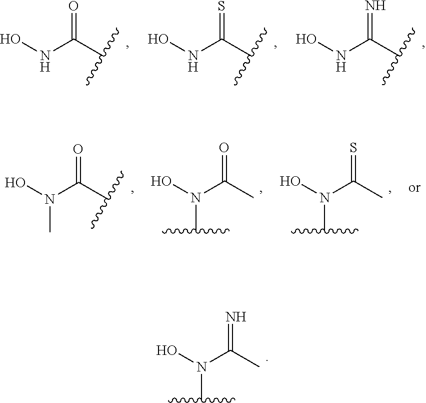

Alternatively, each ligand may, independently, have the following structure

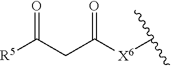

wherein
X⁶ is absent, O, or NR⁶;
R⁵ is optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, optionally substituted C₁₋₈ heteroaryl, —N(R^{5A}) 2, or —OR^{5B};
each R^{5A} is independently hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl;
R^{5B} is hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl; and
R⁶, if present, is hydrogen or optionally substituted C₁₋₆ alkyl.

In particular embodiments, the ligand has any of the following structures:

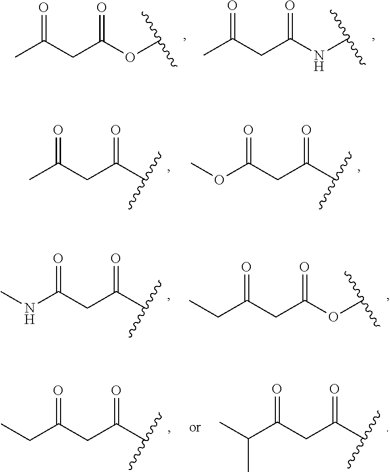

In various embodiments, the ligand may be a carboxylic acid. The carboxylic can be a di-carboxylic acid, a tri-carboxylic acid, or a poly-carboxylic acid. For example, the ligand may be EDTA or an analog thereof, including any of the structures shown below:

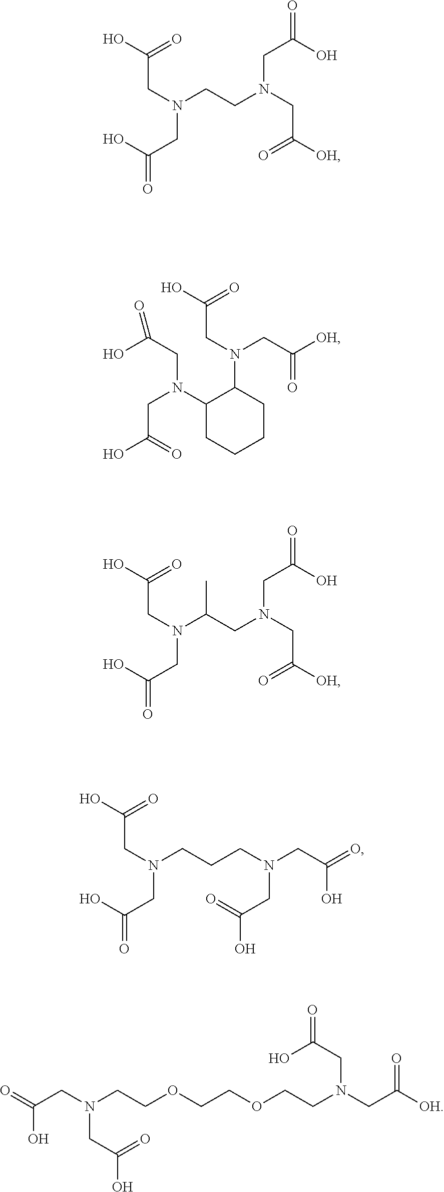 [SMILES: O=C(O)CN(CCN(CC(=O)O)CC(=O)O)CC(=O)O ; O=C(O)CN(CC(=O)O)C1CCCCC1N(CC(=O)O)CC(=O)O ; CC(CN(CC(=O)O)CC(=O)O)N(CC(=O)O)CC(=O)O ; O=C(O)CN(CCCN(CC(=O)O)CC(=O)O)CC(=O)O ; O=C(O)CN(CCOCCOCCN(CC(=O)O)CC(=O)O)CC(=O)O]

### Antibody-Antigen Interactions

The multimeric oligonucleotides of the disclosure may contain one or more noncovalent interactions that is an antibody-antigen interaction. Antibodies are immunoglobulin molecules that specifically bind to, or are immunologically reactive with, a particular antigen, and include polyclonal, monoclonal, genetically engineered and otherwise modified forms of antibodies, including but not limited to chimeric antibodies, humanized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, recombinant IgG (rIgG) fragments, and scFv fragments.

Of particular importance to this binding event is the antigen-binding fragment, which refers to one or more fragments of an antibody that retain the ability to specifically bind to a target antigen. The antigen-binding function of an antibody can be performed by fragments of a full-length antibody. The antibody fragments can be, e.g., a Fab, F(ab′)₂, scFv, SMIP, diabody, a triabody, an affibody, a nanobody, an aptamer, or a domain antibody. Examples of binding fragments of an antibody include, but are not limited to: (i) a Fab fragment, a monovalent fragment consisting of the V_{L}, V_{H}, C_{L}, and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

Antibodies as described above may be useful for forming the multimeric oligonucleotides of the disclosure. For example, there may be one or more siRNA molecules attached to an antibody. This antibody may bind to an antigen, which may contain one or more additional siRNA molecules. Alternatively, the antibody may be capable of binding to more than one antigen (i.e., a multivalent antibody), each of which contains an siRNA molecule, thereby allowing for the noncovalent joining of the two siRNA molecules. Multivalent antibodies are discussed in US Patent Application Nos. 2014/0377269 and 2019/0352401 and U.S. Pat. Nos. 9,758,594 and 10,329,350, the disclosure of each of which is incorporated herein by reference.

The antibody or antigen-binding fragment used herein may be a monoclonal antibody or antigen-binding fragment thereof, a polyclonal antibody or antigen-binding fragment thereof, a human antibody or antigen-binding fragment thereof, a humanized antibody or antigen-binding fragment thereof, a primatized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a nanobody, an antibody-like protein scaffold, a domain antibody, a Fv fragment, a Fab fragment, a F(ab′)₂ molecule, and a tandem scFv (taFv), optionally wherein the antibody or antigen-binding fragment thereof is a human antibody or antigen-binding fragment thereof, a humanized antibody or antigen-binding fragment thereof, or a chimeric antibody or antigen-binding fragment thereof

### Methods of Treatment

The disclosure provides methods of treating a subject in need of gene silencing. The gene silencing may be performed in order to silence defective or overactive genes, silence negative regulators of genes with reduced expression, silence wild type genes with an activating role in a pathway(s) that increases activity of a disease driver gene, silence splice isoforms of a gene(s) that, when selectively knocked down, may elevate total expression of the gene(s), among other reasons, so long as the goal is to restore genetic and biochemical pathway activity from a disease state towards a healthy state. The method may include delivering to the CNS of the subject (e.g., a human) an siRNA molecule of the disclosure or a pharmaceutical composition containing the same by any appropriate route of administration (e.g., intrastriatal, intracerebroventricular, or intrathecal injection). The active compound can be administered in any suitable dose. The actual dosage amount of a composition of the present invention administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, any medical risk(s) associated with a gain of function mutation in the target gene. Subjects that may be treated with the siRNA molecules disclosed herein may include, for example, humans, monkeys, rats, mice, pigs, and other mammals containing at least one orthologous copy of the target gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a multimeric oligonucleotide of the disclosure in admixture with a suitable diluent, carrier, or excipient. The siRNA molecules may be administered, for example, directly into the CNS of the subject (e.g., by way of intracerebroventricular, intrathecal injection or by intra-cisterna magna injection by catheterization).

Conventional procedures and ingredients for the selection and preparation of suitable formulations are described, for example, in Remington, J. P. The Science and Practice of Pharmacy, Easton, PA. Mack Publishers, 2012, 22^{nd} ed. And in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, or by intra-cisterna magna injection by catheterization. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation of Noncovalently Branched siRNA Molecules

This example demonstrates methods of generating non-covalently branched siRNA molecules.

### Nucleic Acid Hybridization Interactions

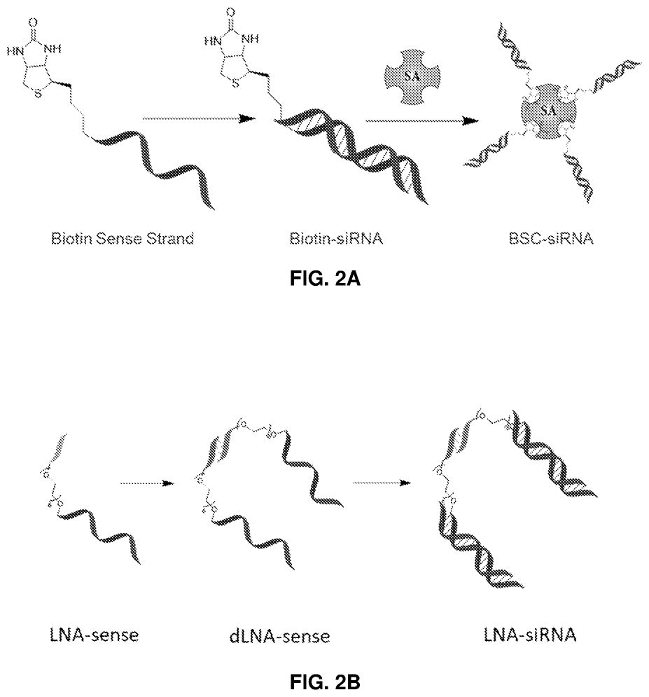

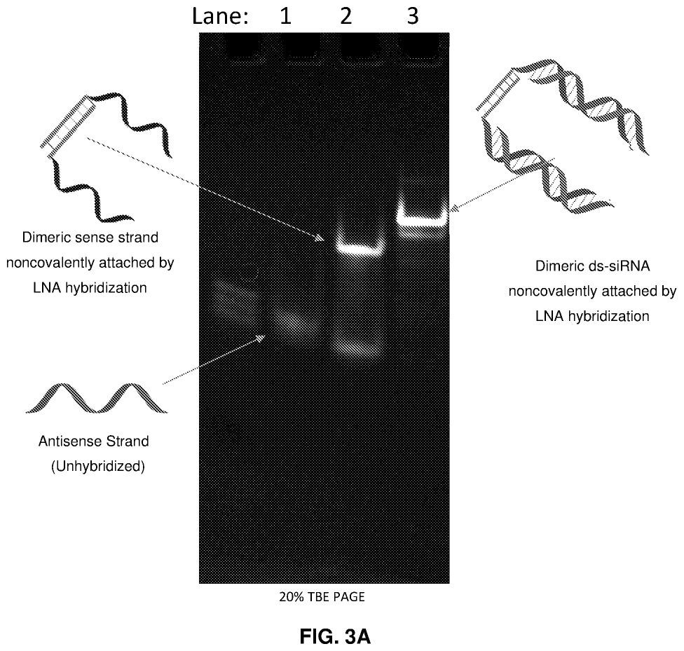

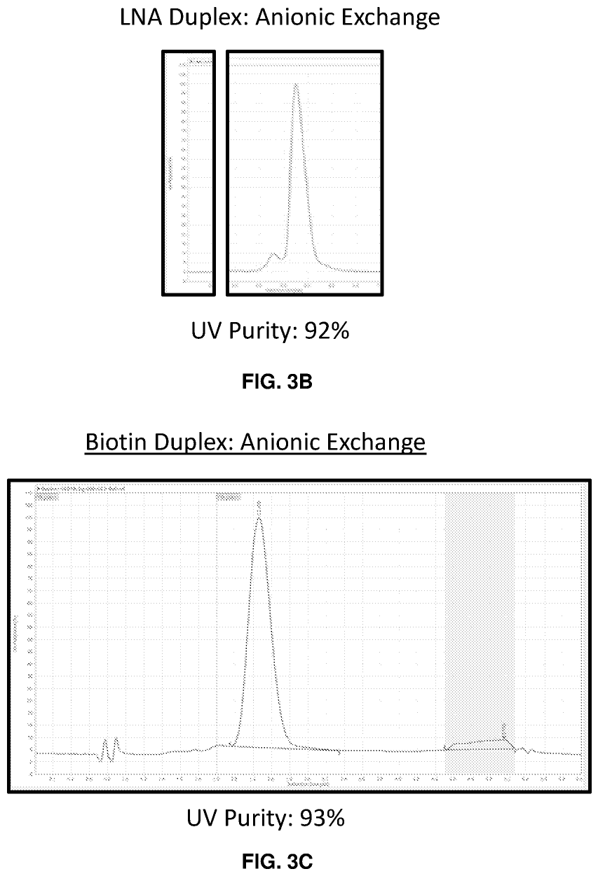

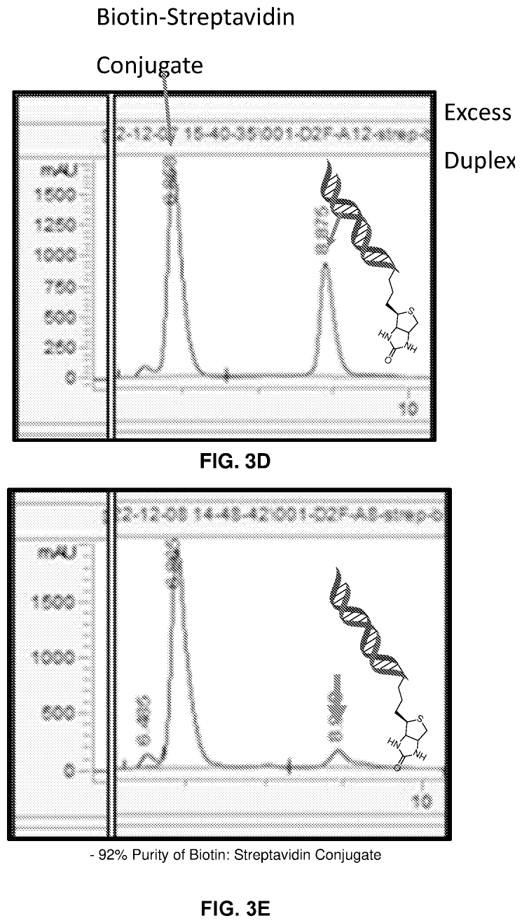

An LNA sequence attached to a sense strand of an siRNA molecule (“LNA-sense”) was designed to have a palindromic sequence of locked nucleic acid (LNA) to generate a dimer of this strand in aqueous solution (“dLNA-sense”). Moreover, this strand was designed with a hexaethylene glycol spacer to maintain flexibility and decrease charge density between the dimerized sense strands. This strand was synthesized, deprotected, purified, desalted, and characterized following standard protocols described previously. Finally, dLNA-sense was hybridized with 2 equiv. of antisense strand to provide the dimeric LNA-siRNA. The resulting compound was characterized by gel electrophoresis (FIG. 3A), and AEX HPLC (FIG. 3B.). A graphical representation of the synthetic sequence is shown in FIG. 2B.

Biotin/Streptavidin Conjugated siRNA

Biotin was first attached to the 3′ end of an siRNA sense strand through oligonucleotide synthesis on biotin functionalized controlled pore glass (CPG). This strand was synthesized, deprotected, purified, desalted, and characterized according to procedures described previously. Biotin siRNA was then generated through the hybridization of the antisense strand with the biotin-sense strand in 1×PBS and characterized by AEX HPLC for UV purity (FIG. 3C). To generate the biotin/streptavidin conjugated-siRNA, biotinylatyed siRNA and streptavidin in 1×PBS were added together in a 4:1 ratio of biotinylated siRNA: streptavidin and analyzed by size exclusion chromatography (SEC) (FIG. 3D). Streptavidin was then further titrated into the solution until the majority of biotin siRNA was bound to streptavidin, as characterized by SEC (FIG. 3E). A graphical representation of the synthetic sequence is shown in FIG. 2A.

### Example 2. Knockdown of HPRT1 mRNA with siRNA Molecules of the Disclosure

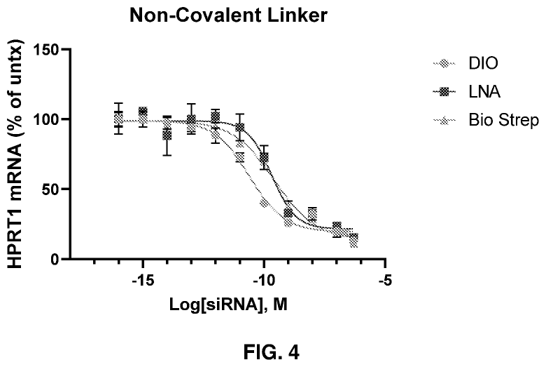

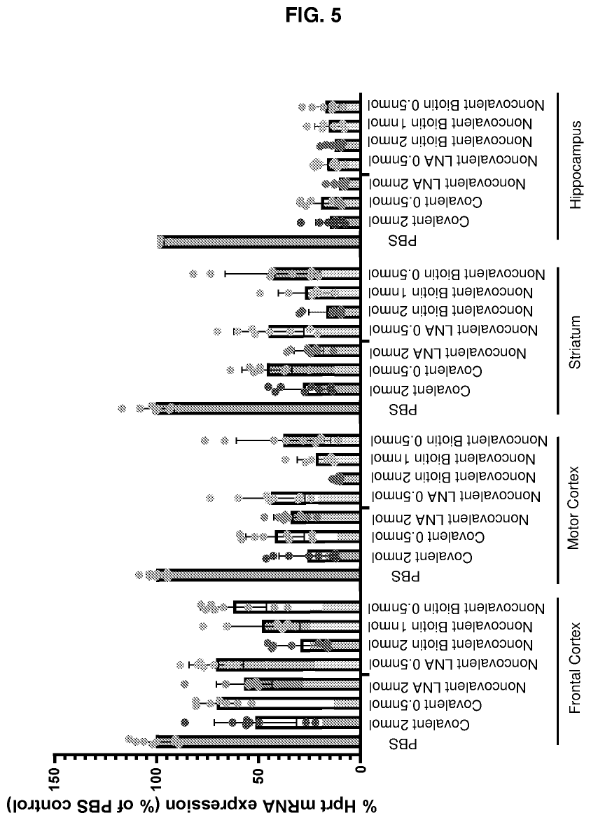

To assess the effects of non-covalently branched siRNA molecules, noncovalently branched SiRNA molecules targeting HPRT1 were delivered into Hela cells by lipid-mediated cellular uptake (RNAiMax). Hela cells were seeded and simultaneously transfected with varied concentrations of the siRNA molecules using RNAiMax. HPRT1 mRNA expression was measured 72 hours post transfection. One siRNA was a dimeric siRNA joined by a nucleic acid hybridization interaction as described in Example 1 (labeled as “LNA” in FIG. 4 and Table 2), a second siRNA was a tetrameric siRNA joined by a biotin/streptavidin interaction as described in example 1 (labeled as “Bio Strep” in FIG. 4 and Table 2), and a third was a covalently attached dimeric siRNA of Formula XVII (labeled as “DIO” in FIG. 4 and Table 2). FIG. 4 and Table 2 demonstrate that each of the siRNA molecules effectively silence HPRT1.

TABLE 2

| Molecule | DIO | LNA | BioStrep |
|---|---|---|---|
| IC50 (pM) | 24.5 | 206 | 264 |
| Number of siRNA subunits | 2 | 2 | 4 |
| per molecule | | | |

### Example 3. In Vivo Evaluation of siRNA Molecules of the Disclosure

FVB/NJ female mice (8-10 weeks old) were treated with LNA hybridized dimeric siRNA (2 or 0.5 nmol) or Biotin-Streptavidin tetrameric siRNA (2, 1 or 0.5 nmol) assembled thru non-covalent bonds. A covalently linked di-branched siRNA molecule of Formula XVII was included in the study (2 or 0.5 nmol) as a positive control. All siRNA molecules were administered to mice (n=8-10/group) by way of a bilateral intracerebroventricular injection. 5 μL was administered per side of the bilateral injection (AP-0.25 mm, ML +/−1 mm, DV-2.5 mm) at a flow rate of 2 μL/minute. Animals were euthanized 28 days post-injection and Hprt mRNA knockdown was quantified by qRT-PCR using Taqman assays. Data was normalized to PBS-treated controls. Table 3, below, shows the conditions tested in this experiment. The ability of each siRNA construct to silence HPRT1 is shown in FIG. 4. These data demonstrate that, surprisingly, noncovalently linked siRNA molecules retain the ability to penetrate the CNS and effect gene silencing. Additionally, the brains from mice treated with Biotin-streptavidin tetrameric siRNA (2 nmol) were stained by either anti-HPRT antibody or microRNAscope probe to detect HPRT protein or the presence of siRNA antisense strand, respectively. Both measurements showed evidence of broad distribution of tetrameric siRNA as well as widespread reduction of HPRT protein throughout all regions of the mouse brain.

TABLE 3

| | | | Relative |
|---|---|---|---|
| | | | Equivalents |
| | | | of siRNA |
| Test Article | Dose | N | monomer |
| PBS | 0 | 10 | — |
| Covalently di-branched siRNA | 2 nmol | 10 | 4 |
| of Formula XVII | | | |
| Covalently di-branched siRNA | 0.5 nmol | 8 | 1 |
| of Formula XVII | | | |
| LNA hybridized dimeric siRNA | 2 nmol | 10 | 4 |
| LNA hybridized dimeric siRNA | 0.5 nmol | 8 | 1 |
| Biotin-streptavidin tetrameric siRNA | 2 nmol | 10 | 8 |
| Biotin-streptavidin tetrameric siRNA | 1 nmol | 6 | 4 |
| Biotin-streptavidin tetrameric siRNA | 0.5 nmol | 8 | 2 |

### Example 4. Method of Preparing a Double-Stranded Short-Interfering RNA Molecule Having Patterned Ribonucleoside Modifications and Internucleoside Linkage Modifications (I)

A double-stranded (ds-) short-interfering (si) RNA (ds-siRNA) molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to established methods (e.g., synthesis and ligation or tandem synthesis) to include alternating patterns (i.e., motifs) of modified ribonucleosides, such as 2′-O-methyl (2′-O-Me) and 2′-fluoro (2′-F) ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

### Antisense Pattern 1 (Formula A1):

A-S-B-S-A-O-B-O-B-O-B-O-A-O-B-O-A-O-B-O-A-O-B-O-A-O-B-O-A-O-B-S-A-S-A-S-A-S-A-S-A

An exemplary sense strand may have any one of the following patterns:

### Sense Pattern 1 (Formula S1):

A-S-A-S-A-O-B-O-A-O-B-O-A-O-B-O-A-O-B-O-A-O-A-O-A-O-B-S-A-S-A

### Sense Pattern 2 (Formula S2):

A-S-A-S-A-O-B-O-A-O-B-O-A-O-B-O-A-O-B-O-A-O-A-O-A-O-B-O-A-O-A

### Sense Pattern 3 (Formula S3):

A-S-A-S-A-O-B-O-A-O-B-O-A-O-B-O-A-O-B-O-A-O-A-O-A-O-B-S-A-S-B

### Sense Pattern 4 (Formula S4):

A-S-A-S-A-O-B-O-A-O-B-O-A-O-B-O-A-O-B-O-A-O-A-O-A-O-B-O-A-O-B

wherein A represents a 2′-O-Me ribonucleoside, B represents a 2′-F ribonucleoside, O represents a phosphodiester internucleoside linkage, and S represents a phosphorothioate internucleoside linkage.

The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 5. Method of Preparing a Ds-siRNA Molecule Having Patterned Ribonucleoside Modifications and Internucleoside Linkage Modifications (II)

A ds-siRNA molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to well-known methods (e.g., synthesis and ligation or tandem synthesis) to contain alternating patterns (i.e., motifs) of modified ribonucleosides, such as 2′-O-Me and 2′-F ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides, 16 nucleotides, 17 nucleotides, 18 nucleotides, 19, nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

### Antisense Pattern 2 (Formula A2):

A-S-B-S-A-O-B-O-A-O-B-O-A-O-B-O-A-O-B-O-A-O-B-O-A-O-B-O-A-O-B-S-A-S-B-S-A-S-A-S-A (FIGS. 9A, 10A, 11A, and 12A);

An exemplary sense strand may have any one of the following patterns:

### Sense Pattern 5 (Formula S5):

A-S-A-S-A-O-B-O-A-O-B-O-A-O-B-O-A-O-B-O-A-O-B-O-A-O-B-S-A-S-A (FIG. 9B);

### Sense Pattern 6 (Formula S6):

A-S-A-S-A-O-B-O-A-O-B-O-A-O-B-O-A-O-B-O-A-O-B-O-A-O-B-O-A-O-A (FIG. 10B)

### Sense Pattern 7 (Formula S7):

A-S-A-S-A-O-B-O-A-O-B-O-A-O-B-O-A-O-B-O-A-O-B-O-A-O-B-S-A-S-B (FIG. 11B);

### Sense Pattern 8 (Formula S8):

A-S-A-S-A-O-B-O-A-O-B-O-A-O-B-O-A-O-B-O-A-O-B-O-A-O-B-O-A-O-B (FIG. 12B)

wherein A represents a 2′-O-Me ribonucleoside, B represents a 2′-F ribonucleoside, O represents a phosphodiester internucleoside linkage, and S represents a phosphorothioate internucleoside linkage.

The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 6. Method of Preparing a Ds-siRNA Molecule Having Patterned Ribonucleoside Modifications and Internucleoside Linkage Modifications (III)

A ds-siRNA molecule having patterned ribonucleoside modifications and internucleoside linkage modifications of the disclosure is prepared according to methods well-known in the art, such as methods disclosed herein. The ds-siRNA molecule is a duplex oligoribonucleotide in which the sense strand is derived from and has full (i.e., 100%) or partial (e.g., at least 70%, 75%, 80%, 85%, 90%, 95%, 96%, 97%, 98%, 99%, or more) sequence identity to an mRNA sequence of a target gene. The nucleic acid sequence of the antisense strand is fully (i.e., 100%) complementary or partially (e.g., at least 70%, 75%, 80%, 85%, 90%, 95%, 96%, 97%, 98%, 99%, or more) complementary to the nucleic acid sequence of the sense strand/mRNA of target gene. The antisense and sense strands of the ds-siRNA agent are each synthesized according to well-known methods (e.g., synthesis and ligation or tandem synthesis) to contain alternating patterns (i.e., motifs) of modified ribonucleosides, such as 2′-O-Me and 2′-F ribonucleosides and modified internucleoside linkages, such as phosphorothioate linkages. The antisense strand is produced to be of a desirable length such that a functional benefit (e.g., RNA interference, thermal stability, and/or resistance against nucleases) is achieved. An exemplary antisense strand may be, e.g., 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. The sense strand is produced to be of a desirable length such that a functional benefit (e.g., efficient RISC loading, thermal stability, and/or resistance against nucleases) is achieved. An exemplary sense strand may be, e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides in length. Consequent to the difference in length between the antisense strand and the sense strand, the ds-siRNA duplex structure contains a 5′ overhang, 3′ overhang, or both. An exemplary antisense strand may have the following pattern:

### Antisense Pattern 3 (Formula A3):

A-S-B-S-A-O-A-O-A-O-B-O-A-O-A-O-A-O-A-O-A-O-A-O-A-O-B-O-A-O-B-S-A-S-A-S-A-S-B-S-A (FIG. 13A);

An exemplary sense strand may have the following pattern:

### Sense Pattern 9 (Formula S9):

A-S-A-S-A-O-A-O-A-O-B-O-B-O-B-O-A-O-B-O-A-O-A-O-A-O-A-S-A-S-A (FIG. 13B)

wherein A represents a 2′-O-Me ribonucleoside, B represents a 2′-F ribonucleoside, O represents a phosphodiester internucleoside linkage, and S represents a phosphorothioate internucleoside linkage. The antisense and sense strand of the ds-siRNA molecule are each produced such that the resulting duplex structure has 0 to 5 nucleotide mismatches (e.g., 0, 1, 2, 3, 4, or 5) mismatches between the sense strand and the antisense strand and/or the antisense strand and the target mRNA sequence. The ds-siRNA molecule may be further modified to incorporate a 5′ phosphorus stabilizing moiety (e.g., a 5′-vinylphosphonate) and/or a hydrophobic moiety (e.g., cholesterol, vitamin D, or tocopherol) on the antisense strand, sense strand, or both. In addition, the ds-siRNA molecule may contain branched structures disclosed herein, such as di-branched, tri-branched, or tetra-branched structures disclosed herein. The ds-siRNA agent may be further incorporated into a pharmaceutical composition containing a pharmaceutically acceptable excipient, carrier, or diluent.

### Example 7. Method of Delivering a Ds-siRNA Molecule to the Central Nervous System of a Patient

A subject, such as a human subject, diagnosed with a disease is treated with a dose and frequency determined by a practitioner (e.g., three times daily, twice daily, once daily, once weekly, once monthly) by administering the siRNA molecule of the disclosure of a pharmaceutical composition containing the same. Dosage and frequency are determined based on the subject's height, weight, age, sex, and other disorders.

A siRNA molecule (e.g., a branched siRNA molecule) having a pattern of chemical modifications disclosed herein is selected by the practitioner for compatibility with the disease and subject. Single- or double-stranded branched siRNA are available for selection. The siRNA chosen has an antisense strand, and in the case of double-stranded siRNA, a sense strand with a sequence and RNA modifications (e.g., natural and non-natural internucleoside linkages, modified sugars, and 5′-phosphorus stabilizing moieties) best suited to the patient and the disease being targeted. For example, the antisense strand may have any one of the antisense strand modification patterns disclosed herein, such as, e.g., Antisense Pattern 1: A-S-B-S-A-O-B-O-B-O-B-O-A-O-B-O-A-O-B-O-A-O-B-O-A-O-B-O-A-O-B-S-A-S-A-S-A-S-A-S-A (Formula A1); Antisense Pattern 2: A-S-B-S-A-O-B-O-A-O-B-O-A-O-B-O-A-O-B-O-A-O-B-O-A-O-B-O-A-O-B-S-A-S-B-S-A-S-A-S-A (Formula A2); or Antisense Pattern 3: A-S-B-S-A-O-A-O-A-O-B-O-A-O-A-O-A-O-A-O-A-O-A-O-A-O-B-O-A-O-B-S-A-S-A-S-A-S-B-S-A (Formula A3). In the case of a ds-siRNA, Antisense Pattern 1 may have a fully or partially complementary sense strand having any one of the patterns of chemical modifications of Sense Pattern 1: A-S-A-S-A-O-B-O-A-O-B-O-A-O-B-O-A-O-B-O-A-O-A-O-A-O-B-S-A-S-A (Formula S1); Sense Pattern 2: A-S-A-S-A-O-B-O-A-O-B-O-A-O-B-O-A-O-B-O-A-O-A-O-A-O-B-O-A-O-A (Formula S2); Sense Pattern 3: A-S-A-S-A-O-B-O-A-O-B-O-A-O-B-O-A-O-B-O-A-O-A-O-A-O-B-S-A-S-B (Formula S3); or Sense Pattern 4: A-S-A-S-A-O-B-O-A-O-B-O-A-O-B-O-A-O-B-O-A-O-A-O-A-O-B-O-A-O-B (Formula S4). In the case of a ds-siRNA having an Antisense Pattern 2, the sense strand may have any one of the patterns of chemical modifications of Sense Pattern 5: A-S-A-S-A-O-B-O-A-O-B-O-A-O-B-O-A-O-B-O-A-O-B-O-A-O-B-S-A-S-A (Formula S5); Sense Pattern 6: A-S-A-S-A-O-B-O-A-O-B-O-A-O-B-O-A-O-B-O-A-O-B-O-A-O-B-O-A-O-A (Formula S6); Sense Pattern 7: A-S-A-S-A-O-B-O-A-O-B-O-A-O-B-O-A-O-B-O-A-O-B-O-A-O-B-S-A-S-B (Formula S7); or Sense Pattern 8: A-S-A-S-A-O-B-O-A-O-B-O-A-O-B-O-A-O-B-O-A-O-B-O-A-O-B-O-A-O-B (Formula S8). In the case of a ds-siRNA having an Antisense Pattern 3, the sense strand may have a sense strand having a pattern of modifications of Sense Pattern 9: A-S-A-S-A-O-A-O-A-O-B-O-B-O-B-O-A-O-B-O-A-O-A-O-A-O-A-S-A-S-A (Formula S9); wherein A and B are different nucleosides (e.g., A is a 2-O-methyl ribonucleoside; B is a 2′-fluoro ribonucleoside), T is phosphorothioate, P is a phosphodiester, and PSM is a 5′-phosphorus stabilizing moiety (e.g., 5′-vinylphosphonate).

The siRNA is delivered by the route best suited the patient and condition (e.g., intrathecally, intracerebroventricularly, or intrastriatally), at a rate tolerable to the patient until the subject has reached a maximum tolerated dose, or until the symptoms of the disease are ameliorated satisfactorily.

### Example 8. Optimizing siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

SIRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

siRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

siRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docohexaenoic acid, docosanoic acid, PC-docosanoic acid, eicosapentaenoic acid, lithocholic acid or any combination of the aforementioned hydrophobic moieties with PC.

SIRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 1, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 9. Preparation and Administrating siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. And in The United States Pharmacopeial Convention, The National Formulary, United States Pharmacopeial, 2015, USP 38 NF 33).

The method of the disclosure contemplates any route of administration to the subject's CNS that is tolerated by the siRNA compositions of the disclosure. Non-limiting examples of siRNA injections into the CNS include intrathecally, intracerebroventricularly, or intra-cisterna magna injection by catheterization. A physician having ordinary skill in the art can readily determine an effective route of administration.

### Example 10. Methods for the Treatment of a Subject in Need of Gene Silencing

A subject in need of gene silencing is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of a target mRNA or suitable biomarker) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the siRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched siRNA, tri-branched siRNA, tetra-branched siRNA, covalently linked siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, or by intra-cisterna magna injection via catheterization) and condition at a rate tolerable to the patient until the subject has reached a maximum tolerated dose, or until symptoms are ameliorated satisfactorily.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A multimeric oligonucleotide comprising from 2 to 8 siRNA molecules that are joined by way of a noncovalent interaction, wherein one or more of the siRNA molecules comprise a noncovalent binding moiety allowing the siRNA molecule to noncovalently bind to at least one other substituent in the multimeric oligonucleotide.

2. The multimeric oligonucleotide of claim 1, wherein each siRNA molecule, independently, comprises a noncovalent binding moiety allowing the siRNA molecule to noncovalently bind to at least one other substituent in the multimeric oligonucleotide.

3. The multimeric oligonucleotide of claim 1 or 2, wherein at least one noncovalent binding moiety comprises a nucleic acid or a nucleic acid analog.

4. The multimeric oligonucleotide of any one of claims 1 to 3, wherein the noncovalent interaction comprises nucleic acid hybridization.

5. The multimeric oligonucleotide of claim 4, wherein the multimeric oligonucleotide comprises the following structure: 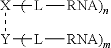
wherein X and Y are each, independently, a nucleic acid or a nucleic acid analog;
each of m and n is, independently, 1, 2, 3, or 4;
each L is, independently, absent or a linker;
each RNA is, independently, an siRNA molecule;
wherein X and Y have complementarity sufficient to hybridize to one another; and
--- represents a hybridization interaction between X and Y.

6. The multimeric oligonucleotide of any one of claims 3 to 5, wherein the nucleic acid comprises DNA nucleosides, RNA nucleosides, or LNA nucleosides.

7. The multimeric oligonucleotide of any one of claims 3 to 6, wherein the nucleic acid comprises LNA nucleosides.

8. The multimeric oligonucleotide of any one of claims 3 to 7, wherein the nucleic acid analog comprises a peptide nucleic acid, a locked nucleic acid, a glycol nucleic acid, a threose nucleic acid, a hexitol nucleic acid, a morpholino oligomer, or a combination thereof.

9. The multimeric oligonucleotide of any one of claims 3 to 8, wherein each nucleic acid comprises from 10 to 100 nucleosides, optionally wherein each nucleic acid comprises from 10 to 90 nucleosides, from 10 to 80 nucleosides, from 10 to 70 nucleosides, from 10 to 60 nucleosides, from 10 to 50 nucleosides, from 10 to 40 nucleosides, from 10 to 30 nucleosides, or from 15 to 30 nucleosides.

10. The multimeric oligonucleotide of any one of claims 1 to 9, wherein the multimeric oligonucleotide comprises from 2 to 7 siRNA molecules.

11. The multimeric oligonucleotide of claim 10, wherein the multimeric oligonucleotide comprises from 2 to 6 siRNA molecules.

12. The multimeric oligonucleotide of claim 11, wherein the multimeric oligonucleotide comprises from 2 to 5 siRNA molecules.

13. The multimeric oligonucleotide of claim 12, wherein the multimeric oligonucleotide comprises from 2 to 4 siRNA molecules.

14. The multimeric oligonucleotide of claim 13, wherein the multimeric oligonucleotide comprises 2 or 3 siRNA molecules.

15. The multimeric oligonucleotide of claim 14, wherein the multimeric oligonucleotide comprises 2 siRNA molecules.

16. The multimeric oligonucleotide of any one of claims 1 to 15, wherein each siRNA molecule is, independently, attached to the noncovalent binding moiety by way of a linker.

17. The multimeric oligonucleotide of any one of claims 1 to 16, wherein the multimeric oligonucleotide comprises the following structure: 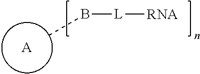
wherein A is a host molecule;
each B is, independently, a guest moiety that noncovalently binds to the host molecule;
each --- is a noncovalent interaction between the host molecule and the guest moiety;
each L is, independently, absent or a linker;
each RNA is, independently, an siRNA molecule; and
n is an integer from 2-8.

18. The multimeric oligonucleotide of claim 17, wherein the multimeric oligonucleotide comprises the following structure:

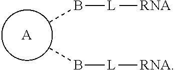

19. The multimeric oligonucleotide of claim 17, wherein the multimeric oligonucleotide comprises the following structure:

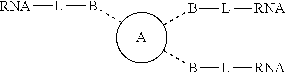

20. The multimeric oligonucleotide of claim 17, wherein the multimeric oligonucleotide comprises the following structure:

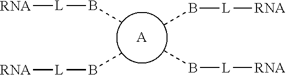

21. The multimeric oligonucleotide of any one of claims 1 to 20, wherein the noncovalent interaction comprises an electrostatic interaction, a Lewis acid-base interaction, a hydrogen-bonding interaction, a hydrophobic interaction, a Van der Waals interaction, a π-effect, or a protein ligand interaction, or a combination thereof.

22. The multimeric oligonucleotide of any one of claims 17 to 21, wherein A is a protein.

23. The multimeric oligonucleotide of claim 22, wherein A is a biotin binding protein

24. The multimeric oligonucleotide of claim 23, wherein A is avidin, streptavidin, NeutrAvidin, Bradavidin II, hoefavidin, rhizavidin, Tamavidin 2, Shwanavidin, Switchavidin, Zebavidin, or Strep-Tactin®.

25. The multimeric oligonucleotide of any one of claims 17 to 24, wherein each B is, independently, a ligand that binds to a protein.

26. The multimeric oligonucleotide of any one of claims 17 to 25, wherein each B is, independently, a peptide.

27. The multimeric oligonucleotide of claim 26, wherein each B has the peptide sequence WSHPQFEK (SEQ ID NO: 1).

28. The multimeric oligonucleotide of any one of claims 17 to 24, wherein each B is, independently: 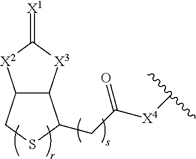
wherein
X¹, X², X³ and X⁴ are each, independently, O or NR²;
each R₂ is, independently, H or optionally substituted C₁₋₆ alkyl;
r is 0 or 1; and
s is 1, 2, 3, 4, 5, or 6.

29. The multimeric oligonucleotide of claim 28, wherein X¹ is O.

30. The multimeric oligonucleotide of claim 28, wherein X¹ is NR².

31. The multimeric oligonucleotide of any one of claims 28 to 30, wherein X² is NR².

32. The multimeric oligonucleotide of any one of claims 28 to 31, wherein X³ is NR².

33. The multimeric oligonucleotide of any one of claims 28 to 32, wherein R² is H.

34. The multimeric oligonucleotide of any one of claims 28 to 32, wherein R² is optionally substituted C₁₋₆ alkyl.

35. The multimeric oligonucleotide of any one of claims 28 to 32 and 34, wherein R² is ethyl.

36. The multimeric oligonucleotide of any one of claims 28 to 30 and 32 to 35, wherein X² is O.

37. The multimeric oligonucleotide of any one of claims 28 to 31 and 33 to 36, wherein X³ is O.

38. The multimeric oligonucleotide of any one of claims 28 to 37, wherein r is 0.

39. The multimeric oligonucleotide of any one of claims 28 to 37, wherein r is 1.

40. The multimeric oligonucleotide of any one of claims 28 to 39, wherein s is 1.

41. The multimeric oligonucleotide of any one of claims 28 to 39, wherein s is 2.

42. The multimeric oligonucleotide of any one of claims 28 to 39, wherein s is 3.

43. The multimeric oligonucleotide of any one of claims 28 to 39, wherein s is 4.

44. The multimeric oligonucleotide of any one of claims 28 to 39, wherein s is 5.

45. The multimeric oligonucleotide of any one of claims 28 to 39, wherein s is 6.

46. The multimeric oligonucleotide of any one of claims 17 to 22, wherein A is a transferrin binding protein.

47. The multimeric oligonucleotide of claim 46, wherein the transferrin binding protein is TbpA or TbpB.

48. The multimeric oligonucleotide of claim 46 or 47, wherein B is transferrin, or an analog thereof, or an isoform thereof.

49. The multimeric oligonucleotide of claim 22, wherein the protein is an antibody or antigen-binding fragment thereof.

50. The multimeric oligonucleotide of claim 49, wherein the antibody is capable of binding to two or more antigens.

51. The multimeric oligonucleotide of any one of claims 17 to 21, 49, and 50, wherein B is an antigen.

52. The multimeric oligonucleotide of any one of claims 17 to 21, wherein A is a metal ion.

53. The multimeric oligonucleotide of claim 52, wherein A is an iron ion, a copper ion, a magnesium ion, a manganese ion, a gadolinium ion, or a zinc ion.

54. The multimeric oligonucleotide of claim 53, wherein A is an iron ion.

55. The multimeric oligonucleotide of claim 53, wherein A is copper (I), copper (II), magnesium (II), manganese (II), manganese (III), manganese (IV), manganese (V), manganese (VI), gadolinium (III), or zinc (II).

56. The multimeric oligonucleotide of claim 55, wherein A is iron (III).

57. The multimeric oligonucleotide of any one of claims 17 to 21 and 52 to 56, wherein each B is, independently: 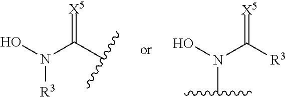
wherein
R³ is hydrogen, optionally substituted C₁-C₆ alkyl, optionally substituted C₁-C₆ alkenyl, optionally substituted C₁-C₆ alkynyl;
X⁵ is O, S, or NR⁴; and
R⁴ is H or optionally substituted C₁₋₆ alkyl.

58. The multimeric oligonucleotide of any one of claims 17 to 21 and 52 to 56, wherein each B is, independently: 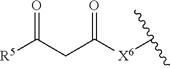
wherein
X⁶ is absent, O, or NR⁶;
R⁵ is optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, optionally substituted C₁₋₈ heteroaryl, —N(R^{5A}) 2, or —OR^{5B};
each R^{5A} is independently hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl;
R^{5B} is hydrogen, optionally substituted C₁₋₆ alkyl, optionally substituted C₁-C₆ alkenyl, optionally substituted C₁₋₆ alkynyl, optionally substituted C₃₋₁₀ cycloalkyl, optionally substituted C₆₋₁₀ aryl, or optionally substituted C₁₋₈ heteroaryl; and
R⁶, if present, is hydrogen or optionally substituted C₁₋₆ alkyl.

59. The multimeric oligonucleotide of any one of claims 5 to 58, wherein the linker is selected from a group consisting of one or more contiguous subunits of an ethylene glycol, alkyl, carbohydrate, block copolymer, peptide, RNA, and DNA.

60. The multimeric oligonucleotide of claim 59, wherein the one or more contiguous subunits is 2 to 20 contiguous subunits.

61. The multimeric oligonucleotide of any one of claims 1 to 60, wherein each siRNA molecule independently comprises an antisense strand and a sense strand having complementarity to the antisense strand, optionally wherein the antisense strand is from 10 to 30 nucleotides in length and has complementarity sufficient to hybridize to a region within a target mRNA.

62. The multimeric oligonucleotide of claim 61, wherein the length of the antisense strand is from 15 to 25 nucleotides.

63. The multimeric oligonucleotide of claim 61 or 62, wherein the length of the antisense strand is 20 nucleotides.

64. The multimeric oligonucleotide of claim 61 or 62, wherein the length of the antisense strand is 21 nucleotides.

65. The multimeric oligonucleotide of claim 61 or 62, wherein the length of the antisense strand is 22 nucleotides.

66. The multimeric oligonucleotide of claim 61 or 62, wherein the length of the antisense strand is 23 nucleotides.

67. The multimeric oligonucleotide of claim 61 or 62, wherein the length of the antisense strand is 24 nucleotides.

68. The multimeric oligonucleotide of claim 61 or 62, wherein the length of the antisense strand is 25 nucleotides.

69. The multimeric oligonucleotide of claim 61, wherein the length of the antisense strand is 26 nucleotides.

70. The multimeric oligonucleotide of claim 61, wherein the length of the antisense strand is 27 nucleotides.

71. The multimeric oligonucleotide of claim 61, wherein the length of the antisense strand is 28 nucleotides.

72. The multimeric oligonucleotide of claim 61, wherein the length of the antisense strand is 29 nucleotides.

73. The multimeric oligonucleotide of claim 61, wherein the length of the antisense strand is 30 nucleotides.

74. The multimeric oligonucleotide of any one of claims 61 to 73, wherein the length of the sense strand is from 12 to 30 nucleotides.

75. The multimeric oligonucleotide of any one of claims 61 to 74, wherein the length of the sense strand is 15 nucleotides.

76. The multimeric oligonucleotide of any one of claims 61 to 74, wherein the length of the sense strand is 16 nucleotides.

77. The multimeric oligonucleotide of any one of claims 61 to 74, wherein the length of the sense strand is 17 nucleotides.

78. The multimeric oligonucleotide of any one of claims 61 to 74, wherein the length of the sense strand is 18 nucleotides.

79. The multimeric oligonucleotide of any one of claims 61 to 74, wherein the length of the sense strand is 19 nucleotides.

80. The multimeric oligonucleotide of any one of claims 61 to 74, wherein the length of the sense strand is 20 nucleotides.

81. The multimeric oligonucleotide of any one of claims 61 to 74, wherein the length of the sense strand is 21 nucleotides.

82. The multimeric oligonucleotide of any one of claims 61 to 74, wherein the length of the sense strand is 22 nucleotides.

83. The multimeric oligonucleotide of any one of claims 61 to 74, wherein the length of the sense strand is 23 nucleotides.

84. The multimeric oligonucleotide of any one of claims 61 to 74, wherein the length of the sense strand is 24 nucleotides.

85. The multimeric oligonucleotide of any one of claims 61 to 74, wherein the length of the sense strand is 25 nucleotides.

86. The multimeric oligonucleotide of any one of claims 61 to 74, wherein the length of the sense strand is 26 nucleotides.

87. The multimeric oligonucleotide of any one of claims 61 to 74, wherein the length of the sense strand is 27 nucleotides.

88. The multimeric oligonucleotide of any one of claims 61 to 74, wherein the length of the sense strand is 28 nucleotides.

89. The multimeric oligonucleotide of any one of claims 61 to 74, wherein the length of the sense strand is 29 nucleotides.

90. The multimeric oligonucleotide of any one of claims 61 to 74, wherein the length of the sense strand is 30 nucleotides.

91. The multimeric oligonucleotide of any one of claims 61 to 90, wherein each antisense strand comprises a structure represented by Formula I, wherein Formula I is, in the 5′-to-3′ direction:
A-B-(A′)ⱼ-C-P²-D-P¹-(C′-P¹)ₖ-C′ Formula I;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

92. The multimeric oligonucleotide of claim 91, wherein each antisense strand comprises a structure represented by Formula A1, wherein Formula A1 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-B-S-A Formula A1;
wherein A represents a 2′-O-Me ribonucleoside, B represents a 2′-F ribonucleoside, O represents a phosphodiester internucleoside linkage, and S represents a phosphorothioate internucleoside linkage.

93. The multimeric oligonucleotide of any one of claims 61 to 90, wherein each antisense strand comprises a structure represented by Formula II, wherein Formula II is, in the 5′-to-3′ direction:
A-B-(A′)ⱼ-C-P²-D-P¹-(C-P¹)ₖ-C′ Formula II;
wherein A is represented by the formula C-P¹-D-P¹;
each A′ is represented by the formula C-P²-D-P²;
B is represented by the formula C-P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

94. The multimeric oligonucleotide of claim 93, wherein each antisense strand comprises a structure represented by Formula A2, wherein Formula A2 is, in the 5′-to-3′ direction:
A-S-B-S-A-O-B-O-B-O-B-O-A-O-B-O-A-O-B-O-A-O-B-O-A-O-B-O-A-O-B-S-A-S-A-S-A-S-A-S-A Formula A2;
wherein A represents a 2′-O-Me ribonucleoside, B represents a 2′-F ribonucleoside, O represents a phosphodiester internucleoside linkage, and S represents a phosphorothioate internucleoside linkage.

95. The multimeric oligonucleotide of any one of claims 61 to 94, wherein each sense strand comprises a structure represented by Formula III, wherein Formula III is, in the 5′-to-3′ direction:
E-(A′)ₘ-F Formula III;
wherein E is represented by the formula (C-P¹)₂;
F is represented by the formula (C-P²)₃-D-P¹-C-P¹-C, (C-P²)₃-D-P²-C-P²-C, (C-P²)₃-D-P¹-C-P¹-D, or (C-P²)₃-D-P²-C-P²-D;
A′, C, D, P¹, and P² are as defined in Formula II; and
m is an integer from 1 to 7.

96. The multimeric oligonucleotide of claim 95, wherein each sense strand comprises a structure represented by Formula S1, wherein Formula S1 is, in the 5′-to-3′ direction:
A-S-A-S-A-O-B-O-A-O-B-O-A-O-B-O-A-O-B-O-A-O-A-O-A-O-B-S-A-S-A Formula S1;
wherein A represents a 2′-O-Me ribonucleoside, B represents a 2′-F ribonucleoside, O represents a phosphodiester internucleoside linkage, and S represents a phosphorothioate internucleoside linkage.

97. The multimeric oligonucleotide of claim 95, wherein each sense strand comprises a structure represented by Formula S2, wherein Formula S2 is, in the 5′-to-3′ direction:
A-S-A-S-A-O-B-O-A-O-B-O-A-O-B-O-A-O-B-O-A-O-A-O-A-O-B-O-A-O-A Formula S2;
wherein A represents a 2′-O-Me ribonucleoside, B represents a 2′-F ribonucleoside, O represents a phosphodiester internucleoside linkage, and S represents a phosphorothioate internucleoside linkage.

98. The multimeric oligonucleotide of claim 95, wherein each sense strand comprises a structure represented by Formula S3, wherein Formula S3 is, in the 5′-to-3′ direction:
A-S-A-S-A-O-B-O-A-O-B-O-A-O-B-O-A-O-B-O-A-O-A-O-A-O-B-S-A-S-B Formula S3;
wherein A represents a 2′-O-Me ribonucleoside, B represents a 2′-F ribonucleoside, O represents a phosphodiester internucleoside linkage, and S represents a phosphorothioate internucleoside linkage.

99. The multimeric oligonucleotide of claim 95, wherein each sense strand comprises a structure represented by Formula S4, wherein Formula S4 is, in the 5′-to-3′ direction:
A-S-A-S-A-O-B-O-A-O-B-O-A-O-B-O-A-O-B-O-A-O-A-O-A-O-B-O-A-O-B Formula S4;
wherein A represents a 2′-O-Me ribonucleoside, B represents a 2′-F ribonucleoside, O represents a phosphodiester internucleoside linkage, and S represents a phosphorothioate internucleoside linkage.

100. The multimeric oligonucleotide of any one of claims 61 to 90 and 95 to 99, wherein each antisense strand comprises a structure represented by Formula IV, wherein Formula IV is, in the 5′-to-3′ direction:
A-(A′)ⱼ-C-P²-B-(C-P¹)ₖ-C′ Formula IV;
wherein A is represented by the formula C-P¹-D-P¹;
each A′ is represented by the formula C-P²-D-P²;
B is represented by the formula D-P¹-C-P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

101. The multimeric oligonucleotide of claim 100, wherein each antisense strand comprises a structure represented by Formula A3, wherein Formula A3 is, in the 5′-to-3′ direction:
A-S-B-S-A-O-B-O-A-O-B-O-A-O-B-O-A-O-B-O-A-O-B-O-A-O-B-O-A-O-B-S-A-S-B-S-A-S-A-S-A Formula A3;
wherein A represents a 2′-O-Me ribonucleoside, B represents a 2′-F ribonucleoside, O represents a phosphodiester internucleoside linkage, and S represents a phosphorothioate internucleoside linkage.

102. The multimeric oligonucleotide of any one of claims 61 to 94, 100, and 101, wherein each sense strand comprises a structure represented by Formula V, wherein Formula V is, in the 5′-to-3′ direction:
E-(A′)ₘ-C-P²-F Formula V;
wherein E is represented by the formula (C-P¹)₂;
F is represented by the formula D-P¹-C-P¹-C, D-P²-C-P²-C, D-P¹-C-P¹-D, or D-P²-C-P²-D;
A′, C, D, P¹ and P² are as defined in Formula IV; and
m is an integer from 1 to 7.

103. The multimeric oligonucleotide of claim 102, wherein each sense strand comprises a structure represented by Formula S5, wherein Formula S5 is, in the 5′-to-3′ direction:
A-S-A-S-A-O-B-O-A-O-B-O-A-O-B-O-A-O-B-O-A-O-B-O-A-O-B-S-A-S-A Formula S5;
wherein A represents a 2′-O-Me ribonucleoside, B represents a 2′-F ribonucleoside, O represents a phosphodiester internucleoside linkage, and S represents a phosphorothioate internucleoside linkage.

104. The multimeric oligonucleotide of claim 102, wherein each sense strand comprises a structure represented by Formula S6, wherein Formula S6 is, in the 5′-to-3′ direction:
A-S-A-S-A-O-B-O-A-O-B-O-A-O-B-O-A-O-B-O-A-O-B-O-A-O-B-O-A-O-A Formula S6;
wherein A represents a 2′-O-Me ribonucleoside, B represents a 2′-F ribonucleoside, O represents a phosphodiester internucleoside linkage, and S represents a phosphorothioate internucleoside linkage.

105. The multimeric oligonucleotide of claim 102, wherein each sense strand comprises a structure represented by Formula S7, wherein Formula S7 is, in the 5′-to-3′ direction:
A-S-A-S-A-O-B-O-A-O-B-O-A-O-B-O-A-O-B-O-A-O-B-O-A-O-B-S-A-S-B Formula S7;
wherein A represents a 2′-O-Me ribonucleoside, B represents a 2′-F ribonucleoside, O represents a phosphodiester internucleoside linkage, and S represents a phosphorothioate internucleoside linkage.

106. The multimeric oligonucleotide of claim 102, wherein each sense strand comprises a structure represented by Formula S8, wherein Formula S8 is, in the 5′-to-3′ direction:
A-S-A-S-A-O-B-O-A-O-B-O-A-O-B-O-A-O-B-O-A-O-B-O-A-O-B-O-A-O-B Formula S8;
wherein A represents a 2′-O-Me ribonucleoside, B represents a 2′-F ribonucleoside, O represents a phosphodiester internucleoside linkage, and S represents a phosphorothioate internucleoside linkage.

107. The multimeric oligonucleotide of any one of claims 61 to 90, 95 to 99, and 102 to 106, wherein each antisense strand comprises a structure represented by Formula VI, wherein Formula VI is, in the 5′-to-3′ direction:
A-Bⱼ-E-Bₖ-E-F-Gₗ-D-P¹-C′ Formula VI;
wherein A is represented by the formula C-P¹-D-P¹;
each B is represented by the formula C-P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²-C-P²;
F is represented by the formula D-P¹-C-P¹;
each G is represented by the formula C-P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

108. The multimeric oligonucleotide of claim 107, wherein each antisense strand comprises a structure represented by Formula A4, wherein Formula A4 is, in the 5′-to-3′ direction:
A-S-B-S-A-O-A-O-A-O-B-O-A-O-A-O-A-O-A-O-A-O-A-O-A-O-B-O-A-O-B-S-A-S-A-S-A-S-B-S-A Formula A4;
wherein A represents a 2′-O-Me ribonucleoside, B represents a 2′-F ribonucleoside, O represents a phosphodiester internucleoside linkage, and S represents a phosphorothioate internucleoside linkage.

109. The multimeric oligonucleotide of any one of claims 61 to 94, 100, 101, 107, and 108, wherein each sense strand comprises a structure represented by Formula VII, wherein Formula VII is, in the 5′-to-3′ direction:
H-Bₘ—Iₙ-A′-Bₒ—H-C Formula VII;
wherein A′ is represented by the formula C-P²-D-P²;
each H is represented by the formula (C-P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

110. The multimeric oligonucleotide of claim 109, wherein each sense strand comprises a structure represented by Formula S9, wherein Formula S9 is, in the 5′-to-3′ direction:
A-S-A-S-A-O-A-O-A-O-B-O-B-O-B-O-A-O-B-O-A-O-A-O-A-O-A-S-A-S-A Formula S9;
wherein A represents a 2′-O-Me ribonucleoside, B represents a 2′-F ribonucleoside, O represents a phosphodiester internucleoside linkage, and S represents a phosphorothioate internucleoside linkage.

111. The multimeric oligonucleotide of any one of claims 61 to 110, wherein each antisense strand further comprises a 5′ phosphorus stabilizing moiety at the 5′ end of the antisense strand.

112. The multimeric oligonucleotide of any one of claims 61 to 111, wherein each sense strand further comprises a 5′ phosphorus stabilizing moiety at the 5′ end of the sense strand.

113. The multimeric oligonucleotide of claim 111 or 112, wherein each 5′ phosphorus stabilizing moiety is, independently, represented by any one of Formulas IX-XVI: 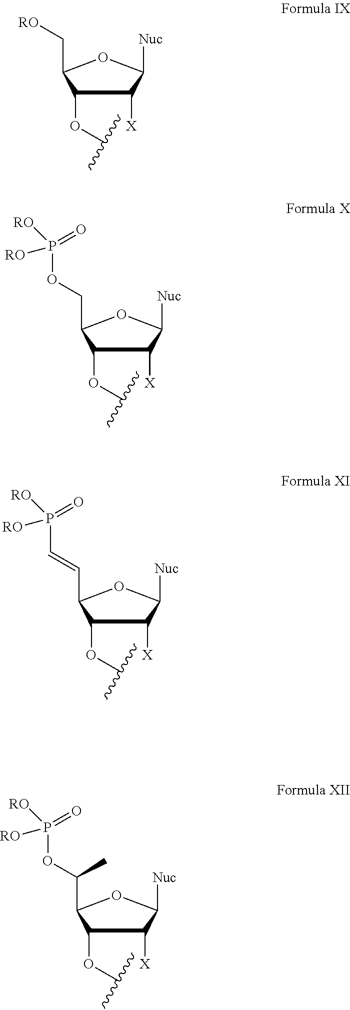 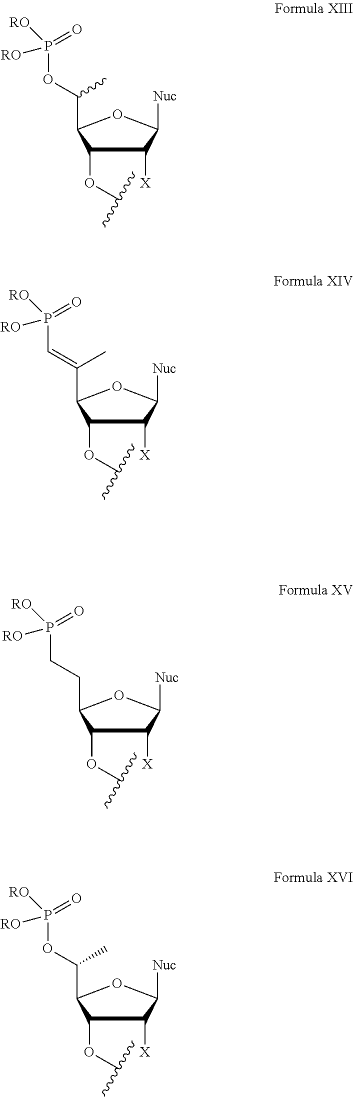
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, hydrogen, or a cation.

114. The multimeric oligonucleotide of any one of claims 111 to 113 wherein the 5′ phosphorus stabilizing moiety is (E)-vinylphosphonate represented by Formula XI.

115. The multimeric oligonucleotide of any one of claims 1 to 114, wherein the siRNA molecule further comprises a hydrophobic moiety at the 5′ or the 3′ end of the siRNA molecule.

116. The multimeric oligonucleotide of claim 115, wherein the hydrophobic moiety is selected from a group consisting of cholesterol, vitamin D, or tocopherol.

117. A composition of siRNA molecules comprising the multimeric oligonucleotide of any one of claims 1 to 116, wherein the multimeric oligonucleotide is present in the composition with a purity of 50% or more.

118. The composition of claim 117, wherein the multimeric oligonucleotide is present in the composition with a purity of 75% or more.

119. The composition of claim 118, wherein the multimeric oligonucleotide is present in the composition with a purity of 80% or more.

120. The composition of claim 119, wherein the multimeric oligonucleotide is present in the composition with a purity of 85% or more.

121. The composition of claim 120, wherein the multimeric oligonucleotide is present in the composition with a purity of 90% or more.

122. The composition of claim 121, wherein the multimeric oligonucleotide is present in the composition with a purity of 95% or more.

123. The composition of claim 122, wherein the multimeric oligonucleotide is present in the composition with a purity of 99% or more.

124. A pharmaceutical composition comprising the multimeric oligonucleotide of any one of claims 1 to 116, or the composition of siRNA molecules of any one of claims 117 to 123, and a pharmaceutically acceptable excipient, carrier, or diluent.

125. A method of delivering an siRNA molecule to the central nervous system (CNS) of a subject, the method comprising administering the multimeric oligonucleotide of any one of claims 1 to 116, or the composition of siRNA molecules of any one of claims 117 to 123, or the pharmaceutical composition of claim 124 to the CNS of the subject.

126. The method of claim 125, wherein the multimeric oligonucleotide, the composition of siRNA molecules, or the pharmaceutical composition is administered to the subject by way of intrastriatal, intracerebroventricular, or intrathecal injection.

127. The method of claim 125 or 126, wherein the delivering of the multimeric oligonucleotide, the composition of siRNA molecules, or the pharmaceutical composition to the CNS of the subject results in gene silencing of a target gene in the subject.

128. The method of claim 127, wherein the target gene is an overactive disease driver gene.

129. The method of claim 127, wherein the target gene is a negative regulator of a gene with reduced expression that is associated with a disease state in the subject.

130. The method of claim 127, wherein the target gene is a positive regulator of a gene with increased expression that is associated with a disease state in a subject.

131. The method of claim 127, wherein the target gene is a splice isoform of the target gene, wherein the splice isoform reduces expression of the target gene.

132. The method of any one of claims 127 to 131, wherein the gene silencing treats a disease state in the subject.

133. The method of any one of claims 125 to 132, wherein the subject is a human.

134. A kit comprising the multimeric oligonucleotide of any one of claims 1 to 116, the composition of siRNA molecules of any one of claims 117 to 123, or the pharmaceutical composition of claim 124, and a package insert, wherein the package insert instructs a user of the kit to perform the method of any one of claims 125 to 133.
